(19) United States
(12) Patent Application Publication
(10) Pub. No.: US 2025/0223247 A1
(43) Pub. Date: Jul. 10, 2025

(54) PROCESS FOR THE HYDROGENATION OF UNSATURATED ESTERS

(71) Applicant: JOHNSON MATTHEY PUBLIC LIMITED COMPANY, London (GB)

(72) Inventors: Damian GRAINGER, London (GB); Krisha VAJARIA, Cambridge (GB)

(21) Appl. No.: 18/850,735

(22) PCT Filed: May 3, 2023
(86) PCT No.: PCT/GB2023/051169

§ 371 Date: Sep. 25, 2024

(30) Foreign Application Priority Data
May 3, 2022 (GB) 2206460.4

(51) Int. Cl. C07C 29/149

(52) U.S. Cl. CPC C07C 29/149

(57) ABSTRACT

Some embodiments relate to a process for the hydrogenation of ester-containing substrates. More specifically, the embodiments relate to a process for the reduction of α,β-γ,δunsaturated containing esters and β,γ unsaturated containing esters of Formula (I) to produce an alcohol of Formula (II). The process of the embodiments uses a base, the conjugate acid of the base has a pKa of from 4 to 15. The process of the embodiments finds use in the synthesis of organic molecules.

### CROSS-REFERENCE TO RELATED APPLICATIONS

This application is a national phase filing under 35 C.F.R. § 371 of and claims priority to PCT Patent Application No. PCT/GB2023/051169, filed on May 3, 2023, which claims the priority benefit under 35 U.S.C. § 119 of British Patent Application No. 2206460.4, filed on May 3, 2022, the contents of which are hereby incorporated in their entireties by reference

### FIELD OF DISCLOSURE

The presently disclosed subject matter relates to a process for the hydrogenation of ester-containing substrates. More specifically, the presently disclosed subject matter relates to a process for the reduction of a,p-y,5 unsaturated containing esters and p,y unsaturated containing esters.

### BACKGROUND

The reduction of esters is an essential transformation in the chemical industry as a route to primary alcohols. The reduction of esters has conventionally been carried out using reagents (in stoichiometric or excess quantities) such as sodium metal in ethanol (the Bouveault-Blanc reduction) or more recently with a metal hydride reagent, such as UAIH4 or NaBH4. These reduction reactions are, however, difficult to carry out effectively on a large scale, not least due to safety concerns associated with an extremely exothermic quenching step. As such, research into the reduction of esters has more recently focussed on catalytic reduction using hydrogen gas. For example, Cu- or Zn-based heterogeneous catalysts are used for ester reduction, primarily in the Natural Detergent Alcohol (NDA) market on very large scale. However, these methods require very high pressures and/or temperatures, in addition to large scale, dedicated production facilities. The chemoselectivity for ester reduction compared to other sensitive functional groups can also be problematic in some cases using these methods.

The reduction of unsaturated esters, such as a,p-y,5 unsaturated esters and p,y unsaturated esters, can be particularly problematic. Finding conditions which are chemoselective for the reduction of the ester group whilst maintaining the regiochemistry of the alkenyl functional group(s) is particularly challenging.

As will be readily understood, a,p,y, and 5, when used in the context of unsaturated esters, refer to the carbon atoms in an ester-containing substrate. a,p,y, and 5 are used to denote the carbon atoms between which an alkene (C═C) or alkyne (C═C) bond is present. Scheme 1 below shows the position of the a,p,y, and 5 carbons in a generic ester-containing substrate; this labelling convention is used herein and throughout. Thus, for example, a p,y unsaturated ester comprises an alkene bond between the p carbon and y carbon, whilst an a,p-y,5 unsaturated ester comprises two alkene bonds; that is an alkene bond between the a carbon and p carbon, and an alkene bond between the y carbon and 5 carbon.

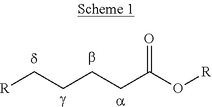

Although a number of processes for ester hydrogenation using transition metal catalysts have been developed, these processes often result in the reduction of alkenyl functional groups, or a change in the regiochemistry of the alkenyl functional groups.

Accordingly, there remains a need for processes capable of selectively reducing unsaturated esters, in particular a, −y, 5 unsaturated esters and p,y unsaturated esters, whilst maintaining the regiochemistry of alkenyl functional groups.

### SUMMARY

Accordingly, the presently disclosed subject matter provides a process for the hydrogenation of an a,p-y,5 unsaturated ester or a p,y unsaturated ester. The process has high chemoselectivity for the hydrogenation of the ester group of a,p-y,5 unsaturated and p,y unsaturated esters to the corresponding alcohol, whilst maintaining the regiochemistry and/or conformational geometry of the alkenyl and/or alkynyl functional group(s).

In a first aspect of the presently disclosed subject matter there is provided a process for the hydrogenation of an ester-containing substrate of Formula (I) to produce an alcohol of Formula (II),

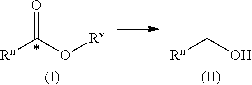

the process comprising treating the ester-containing substrate of Formula (I) with a base and a transition metal catalyst in the presence of molecular hydrogen, wherein:
the ester-containing substrate of Formula (I) comprises an a,p-y,5 unsaturated ester or a p,y unsaturated ester;
R^{u} is an organic group having 3-70 carbon atoms, having the proviso that R^{u} is bonded to the carbonyl carbon (*) of the ester moiety to form the a,p-y,5 unsaturated ester or the p,y unsaturated ester of Formula (I);
R^{v} is an organic group having 1-70 carbon atoms; and
the conjugate acid of the base has a pKa of from 4 to 15.

It is a surprising advantage of the presently disclosed subject matter that the hydrogenation of the a,p-y,5 unsaturated ester or the p,y unsaturated ester proceeds without the reduction of the alkenyl functional group(s) of the R^{u} group and that the regiochemistry and/or conformational geometry of the R^{u} group is maintained as between the ester-containing substrate of Formula (I) and the alcohol of Formula (II). In other words, the R^{u} group is the same in the ester-containing substrate of Formula (I) and in the alcohol of Formula (II). The conjugate acid of the base of the presently disclosed subject matter has a pKa of from 4 to 15, and may be regarded as a weak base. Without wishing to be bound by any sort of theory, it is believed that the use of a base whose conjugate acid has a pKa of from 4 to 15 in the hydrogenation process of the presently disclosed subject matter prevents conjugation of the carbonyl group and alkenyl functional group(s) and minimises or prevents the formation of enolate intermediates. It is speculated that the formation of such enolate intermediates may be responsible for the reduction of the alkenyl functional group(s) and loss of the alkene regiochemistry. Moreover, it is believed that the formation of enolate intermediates may give rise to the formation of other undesirable by-products such as those resulting from cycloaddition reactions (e.g. Diels-Alder) or condensation reactions. It is further surprising that the use of a base whose conjugate acid has a pKa of from 4 to 15 in the hydrogenation process of the presently disclosed subject matter actives and maintains the activity of the transition metal catalyst.

Transition metal catalysed reduction of cinnamate esters, which comprise an a,p unsaturated ester, have been reported (for example, de Vries et. al., Adv. Synth. Catal. 2018, 360). However, conditions which may be suitable for hydrogenating cinnamate esters, may not be suitable for ester-containing substrates of Formula (I). In other words, cinnamate esters do not suffer from the same problems as ester-containing substrate of Formula (I), as described hereinabove, as the present inventors do not believe they form enolate intermediates during hydrogenation processes carried out under basic conditions.

### BRIEF DESCRIPTION OF THE DRAWINGS

FIG. 1 shows an illustrative reaction scheme for the conversion of ester-containing substrate of Formula (I) to a corresponding alcohol of Formula (II).

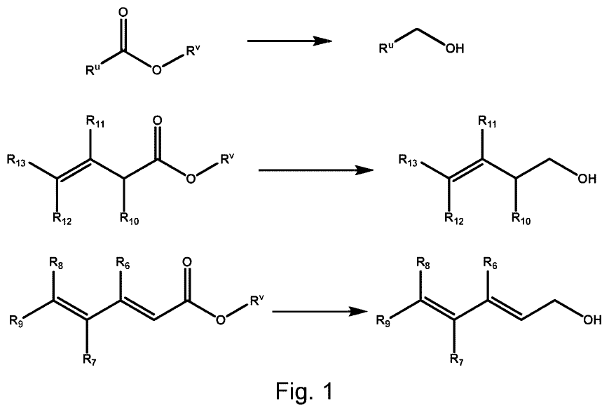

### DEFINITIONS

The point of attachment of a moiety or substituent is represented by “-”. For example, —OH is attached through the oxygen atom.

Unless explicitly stated otherwise, structures depicted herein include all conformational or geometrical isomers of said structure. For example, both E and Z (or c/s and trans) isomers are intended and included where an alkene double bond is shown, and, in the instance where more than one alkene double bond is shown all configurations of conformational or geometrical isomers are included and contemplated (e.g. E,E; E,Z; Z,E; and Z,Z; and cis, cis, cis, trans, trans, cis, and trans, trans).

As used herein, the term “maintenance of regiochemistry” and “retention of regiochemistry” refers to maintenance or retention of the position of any given functional group, for example a p,y alkene bond.

As used here, the term “geometry” is used to refer to the geometric or conformational configuration of a molecule or functional group, for example the conformation of an E or Z (or cis or trans) alkene bond.

As used herein, the term “alkyl” may include straight-chain or branched saturated hydrocarbon groups. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

As used herein, the term “alkenyl” refers to a straight-chain or branched unsaturated hydrocarbon group comprising at least one carbon-carbon double bond.

As used herein, the term “alkynyl” refers to a straight-chain or branched unsaturated hydrocarbon group comprising at least one carbon-carbon triple bond.

As used herein, the term “cycloalkyl” is used to denote a saturated carbocyclic hydrocarbon radical. The cycloalkyl group may have a single ring or multiple condensed rings. In certain embodiments, the cycloalkyl group may have from 3-15 carbon atoms, in certain embodiments, from 3-10 carbon atoms, in certain embodiments, from 3-8 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl, adamantyl and the like.

As used herein, the term “cycloalkenyl” refers to an unsaturated, non-aromatic carbocyclic ring. The cycloalkenyl group therefore has at least one carbon-carbon double bond, but may have more. In certain embodiments, the cycloalkenyl group may have from 3-15 carbon atoms, in certain embodiments, from 3-10 carbon atoms, in certain embodiments, from 3-8 carbon atoms. The cycloalkenyl group may be unsubstituted. Alternatively, the cycloalkenyl group may be substituted. Unless other specified, the cycloalkenyl group may be attached at any suitable carbon atom and, if substituted, may be substituted at any suitable atom. Typical cycloalkenyl groups include but are not limited to cyclopropenyl, cyclobutenyl, cyclopentenyl, cyclohexenyl, and the like.

As used herein, the term “alkoxy” refers to an optionally substituted group of the formula alkyl-O— or cycloalkyl-O—, wherein alkyl and cycloalkyl are as defined above.

As used herein, the term “aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

As used herein, the term “arylalkyl” refers to an optionally substituted group of the formula aryl-alkyl-, where aryl and alkyl are as defined above.

As used herein, the term “halogen”, “halo” or “hal” refers to —F, —Cl, —Br and —I.

As used herein, the term “heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). The heteroalkyl group may be unsubstituted. Alternatively, the heteroalkyl group may be substituted. Unless otherwise specified, the heteroalkyl group may be attached at any suitable atom and, if substituted, may be substituted at any suitable atom. Examples of heteroalkyl groups include but are not limited to ethers, thioethers, primary amines, secondary amines, tertiary amines and the like.

As used herein, the term “heterocycloalkyl” refers to a saturated cyclic hydrocarbon group wherein one or more carbon atoms are independently replaced with one or more heteroatoms (e.g. nitrogen, oxygen, phosphorus and/or sulfur atoms). The heterocycloalkyl group may be unsubstituted. Alternatively, the heterocycloalkyl group may be substituted. Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

As used herein, the term “heteroaryl” refers to an aromatic carbocyclic group wherein one or more carbon atoms are independently replaced with one or more heteroatoms (e.g. nitrogen, oxygen, phosphorus and/or sulfur atoms). The heteroaryl group may be unsubstituted. Alternatively, the heteroaryl group may be substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, tetrazolyl, thiadiazolyl, thiophenyl, oxadiazolyl, pyridinyl, pyrimidyl, benzoxazolyl, benzthiazolyl, benzimidazolyl, indolyl, quinolinyl and the like.

As used herein, the term “heterocycle” encompasses both heterocycloalkyl groups and heteroaryl groups.

As used herein, the term “substituted” refers to a group in which one or more hydrogen atoms are each independently replaced with substituents (e.g. 1, 2, 3, 4, 5 or more) which may be the same or different. Examples of substituents include but are not limited to -halo, —C(halo)3, —R^{c}, ═O, ═S, —O—R^{c}, —S—R^{c}, —NR^{c}R^{d}, —CN, —NO₂, —C(O)—R^{c}, —COOR^{d}, —C(S)—R^{c}, —C(S)OR^{d}, —S(O)₂OH, —S(O)₂—R^{c}, —S(O)₂NR^{c}R^{d}, —O—S(O)—R^{c} and —CONR^{c}R^{d}, such as -halo, —C(halo)₃ (e.g. —CF3), —R^{c}, —O—R^{c}, —NR^{c}R^{d}, —CN, or —NO₂. R^{c} and R^{d} are independently selected from the groups consisting of H, alkyl, aryl, arylalkyl, heteroalkyl, heteroaryl, or R^{c} and R^{d} together with the atom to which they are attached form a heterocycloalkyl group. R^{c} and R^{d} may be unsubstituted or further substituted as defined herein.

As used herein, the term “fatty acid” refers to a carboxylic acid with a long aliphatic chain (e.g. >6 carbon atoms), which can be either saturated or unsaturated. The aliphatic chain of the fatty acid may be branched or unbranched. In certain embodiments, the aliphatic chain of the fatty acid comprises 12 to 24 carbon atoms. In certain embodiments, the aliphatic chain of the fatty acid comprises 0 to 5 carbon-carbon double bonds.

As used herein, the term “fatty alcohol” refers to an alcohol with a long aliphatic chain (e.g. >6 carbon atoms), which can be either saturated or unsaturated. The aliphatic chain of the fatty alcohol may be branched or unbranched. In certain embodiments, the aliphatic chain of the fatty alcohol comprises 12 to 24 carbon atoms. In certain embodiments, the aliphatic chain of the fatty alcohol comprises 0 to 5 carbon-carbon double bonds.

As used herein, the terms “wax ester” or “waxy ester” refer to an ester of a fatty acid and a fatty alcohol, wherein fatty acid and fatty alcohol are as defined above.

As used herein, the term “bidentate ligand” refers to a ligand that donates two pairs of electrons to a metal atom.

As used herein, the term “tridentate ligand” refers to a ligand that donates three pairs of electrons to a metal atom.

As used herein, the term “tetradentate ligand” refers to a ligand that donates four pairs of electrons to a metal atom.

As used herein, the term “Ru—SNS” refers to dichlorotriphenylphosphine[bis(2-(ethylthio)ethyl)amine]ruthenium(ll).

As used herein, the term “Ru—PNN” refers to dichlorotriphenylphosphine[2-(diphenylphosphino)-N-(2-pyridinylmethyl)ethanamine]ruthenium(ll).

As used herein, the term “Ru—SNN” refers to dichlorotriphenylphosphine[2-(ethylthio)-N-(2-pyridinylmethyl)ethanamine]ruthenium(ll).

As used herein, the term “S/C” is an abbreviation for “substrate/catalyst” and is used to describe the catalyst loading employed in a reaction, i.e. it describes the molar ratio of ester-containing substrate and catalyst present in the reaction mixture. In the instance the ester-containing substrate contains more than one ester moiety, the S/C value is adjusted accordingly. For example, a molar ratio of triglyceride to catalyst of 10,000:1 equates to an S/C of 30,000:1 (as a triglyceride substrate contains three ester moieties).

As used herein, the term “turnover number” (TON) refers to the number of moles of substrate that a mole of catalyst can convert before becoming deactivated.

As used herein, unless otherwise specified, “mol %” describes the amount of moles of the specified material (e.g. a base) relative to the amount of moles of the ester-containing substrate, as a percentage. The “mol %” amount given for the specified material (e.g. a base) is the amount of that material employed in the reaction chamber (i.e. the location where the hydrogenation reaction takes place).

As used herein, the term “hydrogenation” refers to hydrogenation using molecular hydrogen.

### DETAILED DESCRIPTION

Preferred and/or optional features of the presently disclosed subject matter will now be set out. Any aspect of the presently disclosed subject matter may be combined with any other aspect of the presently disclosed subject matter unless the context demands otherwise. Any of the preferred and/or optional features of any aspect may be combined, either singly or in combination, with any aspect of the presently disclosed subject matter unless the context demands otherwise.

The presently disclosed subject matter provides a process for the hydrogenation of an ester-containing substrate of Formula (I) to produce an alcohol of Formula (II),

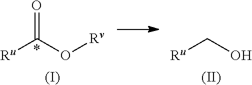

the process comprising treating the ester-containing substrate of Formula (I) with a base and a transition metal catalyst in the presence of molecular hydrogen, wherein:
the ester-containing substrate of Formula (I) comprises an a,p-y,5 unsaturated ester or a p,y unsaturated ester;
R^{u} is an organic group having 3-70 carbon atoms, having the proviso that R^{u} is bonded to the carbonyl carbon (*) of the ester moiety to form the a,p-y,5 unsaturated ester or the p,y unsaturated ester of Formula (I);
R^{v} is an organic group having 1-70 carbon atoms; and
the conjugate acid of the base has a pKa of from 4 to 15.

### Ester-Containing Substrate

The process of the presently disclosed subject matter comprises the hydrogenation of an ester-containing substrate of Formula (I), wherein the ester-containing substrate of Formula (I) comprises an a,p-y,5 unsaturated ester or a p,y unsaturated ester. The ester-containing substrate of Formula (I) contains at least one ester moiety.

In preferred processes of the presently disclosed subject matter the ester-containing substrate of Formula (I) comprises an a,p-y,5 unsaturated ester. In preferred processes of the presently disclosed subject matter the ester-containing substrate of Formula (I) comprises a p,y unsaturated ester.

In preferred processes of the presently disclosed subject matter the ester-containing substrate of Formula (I) comprises an a,p-y,5 unsaturated ester or a p,y unsaturated ester and the bond between the a and p carbon atoms and the y and 5 carbon atoms in the a,p-y,5 unsaturated ester, or the p and y carbon atoms in the p,y unsaturated ester are each alkenyl bonds. In the instance where the ester-containing substrate of Formula (I) is an a,p-y,5 unsaturated ester, it may be preferred that the a,p-y,5 unsaturated ester has a cis, cis geometry, a cis, trans geometry, a trans, cis geometry, or a trans, trans geometry. In the instance where the ester-containing substrate of Formula (I) is a p,y unsaturated ester, it may be preferred that the p,y unsaturated ester has a cis geometry or a trans geometry.

The ester-containing substrate is of Formula (I):

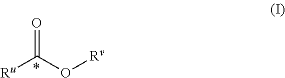

wherein:
R^{u} is an organic group having 3-70 carbon atoms, having the proviso that R^{u} is bonded to the carbonyl carbon (*) of the ester moiety to form the a,p-5,y unsaturated ester or the p, 8 unsaturated ester of Formula (I); and
R^{v} is an organic group having 1-70 carbon atoms.

As will be understood, R^{u} is bonded to the carbonyl carbon (*) of the ester moiety of the ester-containing substrate of Formula (I) to form the a,p-y,5 unsaturated ester or the p,y unsaturated ester.

In preferred processes of the presently disclosed subject matter, R^{u} does not comprise a terminal CH2 group having a sp² hybridised carbon atom. Thus, in preferred processes of the presently disclosed subject matter, there is the proviso that R^{u} is bonded to the carbonyl carbon (*) of the ester moiety to form the a,p-5,y unsaturated ester or the p,8 unsaturated ester of Formula (I), and R^{u} does not comprise a terminal CH2 group having a sp² hybridised carbon atom.

Preferably, R^{u} is an organic group having Formula (XII):

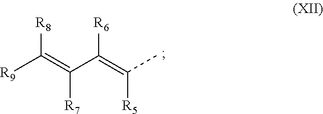

wherein the dashed bond indicates the bond to the carbonyl carbon (*) of the ester moiety of the ester-containing substrate of Formula (I).

In the instance where R^{u} is an organic group having Formula (XII) the ester-containing substrate of Formula (I) comprises an a,p-y,5 unsaturated ester.

Rs-Rs are each independently a hydrogen atom or an organic group having 1-70 carbon atoms.

In preferred processes of the presently disclosed subject matter, Rs-Rs are each independently selected from a hydrogen atom, substituted or unsubstituted Ci-70-alkyl, substituted or unsubstituted C2-70-alkenyl, substituted or unsubstituted C2-70-alkynyl, substituted or unsubstituted C1·70-heteroalkyl, substituted or unsubstituted Cs-70-cycloalkyl, substituted or unsubstituted C3-70-cycloalkenyl, substituted or unsubstituted C2-70-heterocycloalkyl, substituted or unsubstituted Ce-70-aryl, and substituted or unsubstituted C4-7o-heteroaryl, preferably substituted or unsubstituted Ci-50-alkyl, substituted or unsubstituted C2-5o-alkenyl, substituted or unsubstituted C2-5o-alkynyl, substituted or unsubstituted Ci-so-heteroalkyl, substituted or unsubstituted Cs-so-cycloalkyl, substituted or unsubstituted Cs-so-cycloalkenyl, substituted or unsubstituted C2-50-heterocycloalkyl, substituted or unsubstituted Ce-so-aryl, and substituted or unsubstituted C4-50-heteroaryl, more preferably substituted or unsubstituted Ci-3o-alkyl, substituted or unsubstituted C2-3o-alkenyl, substituted or unsubstituted C2-3o-alkynyl, substituted or unsubstituted Ci-30-heteroalkyl, substituted or unsubstituted Cs-so-cycloalkyl, substituted or unsubstituted Cs-so-cycloalkenyl, substituted or unsubstituted C2-30-heterocycloalkyl, substituted or unsubstituted Ce-so-aryl, and substituted or unsubstituted C4-30-heteroaryl, even more preferably substituted or unsubstituted Ci-20-alkyl (e.g. Cs-2o-alkyl), substituted or unsubstituted C2-2o-alkenyl (e.g. Cs-2o-alkenyl), substituted or unsubstituted C2-20-alkynyl (e.g. Cs-20-alkynyl), substituted or unsubstituted Ci-20-heteroalkyl (e.g. Cs-20-heteroalkyl), substituted or unsubstituted C3-2o-cycloalkyl (e.g. Cs-20-cycloalkyl), substituted or unsubstituted C3-2o-cycloalkenyl (e.g. Cs-20-cycloalkenyl), substituted or unsubstituted C2-20-heterocycloalkyl (e.g. C8-20-heterocycloalkyl), substituted or unsubstituted C6-2o-aryl (e.g. Cs-20-aryl), and substituted or unsubstituted C4-2o-heteroaryl (e.g. Cs-20-heteroaryl). Preferably, R5-R8 are each independently selected from a hydrogen atom, substituted or unsubstituted Ci-70-alkyl, substituted or unsubstituted C2-7o-alkenyl, substituted or unsubstituted C1·70-heteroalkyl, substituted or unsubstituted C6-7o-aryl, and substituted or unsubstituted C4-70-heteroaryl, more preferably substituted or unsubstituted Ci-50-alkyl, substituted or unsubstituted C2-5o-alkenyl, substituted or unsubstituted Ci-so-heteroalkyl, substituted or unsubstituted Ce-so-aryl, and substituted or unsubstituted C4-5o-heteroaryl, even more preferably substituted or unsubstituted Ci-so-alkyl, substituted or unsubstituted C2-3o-alkenyl, substituted or unsubstituted Ci-3o-heteroalkyl, substituted or unsubstituted Ce-so-aryl, and substituted or unsubstituted C4-3o-heteroaryl, even more preferably substituted or unsubstituted Ci-20-alkyl (e.g. Cs-2o-alkyl), substituted or unsubstituted C2-2o-alkenyl (e.g. Cs-20-alkenyl), substituted or unsubstituted Ci-20-heteroalkyl (e.g. Cs-20-heteroalkyl), substituted or unsubstituted Ce-2o-aryl (e.g. Cs-20-aryl), and substituted or unsubstituted C4-2o-heteroaryl (e.g. Cs-20-heteroaryl). More preferably, Rs-Rs are each independently selected from a hydrogen atom, substituted or unsubstituted Ci-70-alkyl, substituted or unsubstituted C2-7o-alkenyl, and substituted or unsubstituted C6-7o-aryl, more preferably a hydrogen atom, substituted or unsubstituted Ci-50-alkyl, substituted or unsubstituted C2-5o-alkenyl, and substituted or unsubstituted Ce-so-aryl, even more preferably a hydrogen atom, substituted or unsubstituted Ci-30-alkyl, substituted or unsubstituted C2-3o-alkenyl, and substituted or unsubstituted Ce-so-aryl, even more preferably a hydrogen atom, substituted or unsubstituted Ci-20-alkyl (e.g. Cs-20-alkyl), substituted or unsubstituted C2-2o-alkenyl (e.g. Cs-2o-alkenyl), and substituted or unsubstituted Ce-2o-aryl (e.g. Cs-2o-aryl). More preferably, Rs-Rs are each independently selected from a hydrogen atom, substituted or unsubstituted C o-alkyl and substituted or unsubstituted Ce-70-aryl, more preferably a hydrogen atom, substituted or unsubstituted Ci-so-alkyl and substituted or unsubstituted Ce-so-aryl, even more preferably a hydrogen atom, substituted or unsubstituted Ci-3o-alkyl and substituted or unsubstituted Ce-so-aryl, even more preferably a hydrogen atom, substituted or unsubstituted Ci-2o-alkyl (e.g. Cs-2o-alkyl) and substituted or unsubstituted C6-2o-aryl (e.g. Cs-2o-aryl). Even more preferably, Rs-Rs are each independently selected from a hydrogen atom, methyl, ethyl, n-propyl, iso-propyl, n-butyl, secbutyl, tert-butyl, pentyl, hexyl, heptyl, octyl, phenyl, tolyl, xylyl, and methoxyphenyl. Most preferably, Rs-Rs are hydrogen atoms. Alternatively, two of Rs, Re, and R? or two of Re, R7, and Rs together with the atoms to which they are attached form a substituted or unsubstituted cycloalkyl ring, a substituted or unsubstituted cycloalkenyl ring, or a substituted or unsubstituted heterocycloalkyl ring, for example an alkyl cyclohex-2-ene-1-carboxylate or an alkyl cyclopentene-1-carboxylate.

Rg is H or an organic group having 1-70 carbon atoms. Preferably, Rg is H, a substituted or unsubstituted Ci-70-alkyl, substituted or unsubstituted C2-7o-alkenyl, substituted or unsubstituted C2-7o-alkynyl, substituted or unsubstituted C o-heteroalkyl, substituted or unsubstituted C3-7o-cycloalkyl, substituted or unsubstituted C3-7o-cycloalkenyl, substituted or unsubstituted C2-70-heterocycloalkyl, substituted or unsubstituted C6-70-aryl, and substituted or unsubstituted C4-7o-heteroaryl, preferably H, substituted or unsubstituted Ci-50-alkyl, substituted or unsubstituted C2-5o-alkenyl, substituted or unsubstituted C2-5o-alkynyl, substituted or unsubstituted Ci-so-heteroalkyl, substituted or unsubstituted Cs-so-cycloalkyl, substituted or unsubstituted Cs-so-cycloalkenyl, substituted or unsubstituted C2-50-heterocycloalkyl, substituted or unsubstituted Ce-so-aryl, and substituted or unsubstituted C4-50-heteroaryl, more preferably H, substituted or unsubstituted Ci-30-alkyl, substituted or unsubstituted C2-3o-alkenyl, substituted or unsubstituted C2-3o-alkynyl, substituted or unsubstituted Ci-3o-heteroalkyl, substituted or unsubstituted Cs-so-cycloalkyl, substituted or unsubstituted Cs-so-cycloalkenyl, substituted or unsubstituted C2-30-heterocycloalkyl, substituted or unsubstituted Ce-so-aryl, and substituted or unsubstituted C4-3o-heteroaryl, even more preferably H, substituted or unsubstituted Ci-20-alkyl (e.g. Cs-2o-alkyl), substituted or unsubstituted C2-2o-alkenyl (e.g. Cs-2o-alkenyl), substituted or unsubstituted C2-2o-alkynyl (e.g. Cs-20-alkynyl), substituted or unsubstituted Ci-20-heteroalkyl (e.g. Cs-20-heteroalkyl), substituted or unsubstituted Cs-20-cycloalkyl (e.g. Cs-20-cycloalkyl), substituted or unsubstituted C3-20-cycloalkenyl (e.g. Cs-20-cycloalkenyl), substituted or unsubstituted C2-20-heterocycloalkyl (e.g. Cs-20-heterocycloalkyl), substituted or unsubstituted C6-2o-aryl (e.g. Cs-2o-aryl), and substituted or unsubstituted C4-2O-heteroaryl (e.g. Cs-20-heteroaryl). Preferably, Rg is selected from H, substituted or unsubstituted Ci-70-alkyl, substituted or unsubstituted C2-7o-alkenyl, substituted or unsubstituted C o-heteroalkyl, substituted or unsubstituted C6-7o-aryl, and substituted or unsubstituted C4-7o-heteroaryl, more preferably H, substituted or unsubstituted Ci-50-alkyl, substituted or unsubstituted C2-5o-alkenyl, substituted or unsubstituted Ci-50-heteroalkyl, substituted or unsubstituted Ce-so-aryl, and substituted or unsubstituted C4-5o-heteroaryl, even more preferably H, substituted or unsubstituted Ci-30-alkyl, substituted or unsubstituted C2-30-alkenyl, substituted or unsubstituted Ci-30-heteroalkyl, substituted or unsubstituted Ce-so-aryl, and substituted or unsubstituted C4-3o-heteroaryl, even more preferably H, substituted or unsubstituted Ci-2o-alkyl (e.g. Cs-2o-alkyl), substituted or unsubstituted C2-2o-alkenyl (e.g. Cs-20-alkenyl), substituted or unsubstituted Ci-20-heteroalkyl (e.g. Cs-20-heteroalkyl), substituted or unsubstituted C6-2o-aryl (e.g. Cs-2o-aryl), and substituted or unsubstituted C4-2o-heteroaryl (e.g. Cs-20-heteroaryl). More preferably, Rg is selected from H, substituted or unsubstituted C1·70-alkyl, substituted or unsubstituted C2-7o-alkenyl, and substituted or unsubstituted C6-7o-aryl, more preferably H, substituted or unsubstituted Ci-50-alkyl, substituted or unsubstituted C2-50-alkenyl, and substituted or unsubstituted Ce-so-aryl, even more preferably H, substituted or unsubstituted Ci-30-alkyl, substituted or unsubstituted C2-3o-alkenyl, and substituted or unsubstituted Ce-so-aryl, even more preferably H, substituted or unsubstituted Ci-20-alkyl (e.g. Cs-20-alkyl), substituted or unsubstituted C2-2o-alkenyl (e.g. Cs-2o-alkenyl), and substituted or unsubstituted Ce-2o-aryl (e.g. Cs-20-aryl). More preferably, Rg is selected from H, a substituted or unsubstituted C o-alkyl and substituted or unsubstituted Ce-7o-aryl, more preferably H, substituted or unsubstituted Ci-50-alkyl and substituted or unsubstituted Ce-so-aryl, even more preferably H, substituted or unsubstituted Ci-30-alkyl and substituted or unsubstituted Ce-so-aryl, even more preferably H, substituted or unsubstituted Ci-2o-alkyl (e.g. Cs-2o-alkyl) and substituted or unsubstituted Ce-2o-aryl (e.g. Cs-2o-aryl). Even more preferably, Rg is selected from H, methyl, ethyl, n-propyl, iso-propyl, n-butyl, sec-butyl, tert-butyl, pentyl, hexyl, heptyl, octyl, phenyl, tolyl, xylyl, and methoxyphenyl. Most preferably, Rg is selected from methyl, ethyl, and n-propyl.

Alternatively, R^{u} may be an organic group having Formula (XIII):

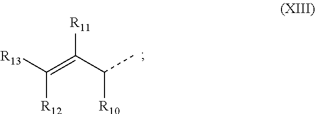

wherein the dashed bond indicates the bond to the carbonyl carbon (*) of the ester moiety of the ester-containing substrate of Formula (I).
In the instance where R^{u} is an organic group of Formula (XIII) the ester-containing substrate of Formula (I) comprises a p,y unsaturated ester.

R—Ri2 are each independently a hydrogen atom or an organic group having 1-70 carbon atoms.

In preferred processes of the presently disclosed subject matter, R10-R12 are each independently selected from a hydrogen atom, substituted or unsubstituted Ci-70-alkyl, substituted or unsubstituted C2-70-alkenyl, substituted or unsubstituted C2-7o-alkynyl, substituted or unsubstituted C1·70-heteroalkyl, substituted or unsubstituted Cs-70-cycloalkyl, substituted or unsubstituted C3-70-cycloalkenyl, substituted or unsubstituted C2-70-heterocycloalkyl, substituted or unsubstituted Ce-70-aryl, and substituted or unsubstituted C4-7o-heteroaryl, preferably substituted or unsubstituted Ci-50-alkyl, substituted or unsubstituted C2-5o-alkenyl, substituted or unsubstituted C2-5o-alkynyl, substituted or unsubstituted Ci-so-heteroalkyl, substituted or unsubstituted Cs-so-cycloalkyl, substituted or unsubstituted Cs-so-cycloalkenyl, substituted or unsubstituted C2-50-heterocycloalkyl, substituted or unsubstituted Ce-so-aryl, and substituted or unsubstituted C4-5o-heteroaryl, more preferably substituted or unsubstituted Ci-30-alkyl, substituted or unsubstituted C2-3o-alkenyl, substituted or unsubstituted C2-3o-alkynyl, substituted or unsubstituted Ci-3o-heteroalkyl, substituted or unsubstituted Cs-so-cycloalkyl, substituted or unsubstituted Cs-so-cycloalkenyl, substituted or unsubstituted C2-30-heterocycloalkyl, substituted or unsubstituted Ce-so-aryl, and substituted or unsubstituted C4-30-heteroaryl, even more preferably substituted or unsubstituted Ci-20-alkyl (e.g. Cs-2o-alkyl), substituted or unsubstituted C2-2o-alkenyl (e.g. Cs-2o-alkenyl), substituted or unsubstituted C2-20-alkynyl (e.g. Cs-20-alkynyl), substituted or unsubstituted Ci-20-heteroalkyl (e.g. Cs-20-heteroalkyl), substituted or unsubstituted Cs-20-cycloalkyl (e.g. Cs-20-cycloalkyl), substituted or unsubstituted C3-2o-cycloalkenyl (e.g. Cs-20-cycloalkenyl), substituted or unsubstituted C2-20-heterocycloalkyl (e.g. Cs-20-heterocycloalkyl), substituted or unsubstituted C6-2o-aryl (e.g. Cs-20-aryl), and substituted or unsubstituted C4-2o-heteroaryl (e.g. Cs-20-heteroaryl). Preferably, R10-R12 are each independently selected from a hydrogen atom, substituted or unsubstituted Ci-70-alkyl, substituted or unsubstituted C2-7o-alkenyl, substituted or unsubstituted C1·70-heteroalkyl, substituted or unsubstituted C6-7o-aryl, and substituted or unsubstituted C4-70-heteroaryl, more preferably substituted or unsubstituted Ci-50-alkyl, substituted or unsubstituted C2-5o-alkenyl, substituted or unsubstituted Ci-50-heteroalkyl, substituted or unsubstituted Ce-so-aryl, and substituted or unsubstituted C4-5o-heteroaryl, even more preferably substituted or unsubstituted Ci-3o-alkyl, substituted or unsubstituted C2-3o-alkenyl, substituted or unsubstituted Ci-3o-heteroalkyl, substituted or unsubstituted Ce-so-aryl, and substituted or unsubstituted C4-3o-heteroaryl, even more preferably substituted or unsubstituted Ci-2o-alkyl (e.g. Cs-2o-alkyl), substituted or unsubstituted C2-2o-alkenyl (e.g. Cs-20-alkenyl), substituted or unsubstituted Ci-20-heteroalkyl (e.g. Cs-20-heteroalkyl), substituted or unsubstituted Ce-2o-aryl (e.g. Cs-2o-aryl), and substituted or unsubstituted C4-2o-heteroaryl (e.g.

Cs-20-heteroaryl)—More preferably, R10-R12 are each independently selected from a hydrogen atom, substituted or unsubstituted Ci-70-alkyl, substituted or unsubstituted C2-7o-alkenyl, and substituted or unsubstituted Ce-70-aryl, more preferably a hydrogen atom, substituted or unsubstituted Ci-50-alkyl, substituted or unsubstituted C2-5o-alkenyl, and substituted or unsubstituted Ce-so-aryl, even more preferably a hydrogen atom, substituted or unsubstituted Ci-30-alkyl, substituted or unsubstituted C2-3o-alkenyl, and substituted or unsubstituted Ce-so-aryl, even more preferably a hydrogen atom, substituted or unsubstituted Ci-20-alkyl (e.g. Cs-20-alkyl), substituted or unsubstituted C2-2o-alkenyl (e.g. Cs-2o-alkenyl), and substituted or unsubstituted Ce-2o-aryl (e.g. Cs-2o-aryl). More preferably, R10-R12 are each independently selected from a hydrogen atom, substituted or unsubstituted C o-alkyl and substituted or unsubstituted C6-7o-aryl, more preferably a hydrogen atom, substituted or unsubstituted C1·50-alkyl and substituted or unsubstituted Ce-so-aryl, even more preferably a hydrogen atom, substituted or unsubstituted Ci-so-alkyl and substituted or unsubstituted Ce-so-aryl, even more preferably a hydrogen atom, substituted or unsubstituted Ci-2o-alkyl (e.g. Cs-2o-alkyl) and substituted or unsubstituted Ce-2o-aryl (e.g. Cs-2o-aryl). Even more preferably, R10-R12 are each independently selected from a hydrogen atom, methyl, ethyl, n-propyl, iso-propyl, n-butyl, secbutyl, tert-butyl, pentyl, hexyl, heptyl, octyl, phenyl, tolyl, xylyl, and methoxyphenyl. Most preferably, R10-R12 are hydrogen atoms. Alternatively, two of R10, R11, and R12 together with the atoms to which they are attached form a substituted or unsubstituted cycloalkyl ring, a substituted or unsubstituted cycloalkenyl ring, or a substituted or unsubstituted heterocycloalkyl ring.

R13 is H or an organic group having 1-70 carbon atoms. Preferably, R13 is H, a substituted or unsubstituted Ci-70-alkyl, substituted or unsubstituted C2-7o-alkenyl, substituted or unsubstituted C2-7o-alkynyl, substituted or unsubstituted C o-heteroalkyl, substituted or unsubstituted C3-7o-cycloalkyl, substituted or unsubstituted C3-7o-cycloalkenyl, and substituted or unsubstituted C2-70-heterocycloalkyl, preferably H, substituted or unsubstituted Ci-50-alkyl, substituted or unsubstituted C2-5o-alkenyl, substituted or unsubstituted C2-5o-alkynyl, substituted or unsubstituted Ci-so-heteroalkyl, substituted or unsubstituted Cs-so-cycloalkyl, substituted or unsubstituted Cs-so-cycloalkenyl, and substituted or unsubstituted C2-50-heterocycloalkyl, more preferably H, substituted or unsubstituted Ci-30-alkyl, substituted or unsubstituted C2-3o-alkenyl, substituted or unsubstituted C2-3o-alkynyl, substituted or unsubstituted Ci-3o-heteroalkyl, substituted or unsubstituted Cs-so-cycloalkyl, substituted or unsubstituted Cs-so-cycloalkenyl, and substituted or unsubstituted C2-30-heterocycloalkyl, even more preferably H, substituted or unsubstituted Ci-2o-alkyl (e.g. Cs-2o-alkyl), substituted or unsubstituted C2-2o-alkenyl (e.g. Cs-2o-alkenyl), substituted or unsubstituted C2-2o-alkynyl (e.g. Cs-20-alkynyl), substituted or unsubstituted Ci-2o-heteroalkyl (e.g. Cs-20-heteroalkyl), substituted or unsubstituted C3-2o-cycloalkyl (e.g. Cs-20-cycloalkyl), substituted or unsubstituted C3-2o-cycloalkenyl (e.g. Cs-20-cycloalkenyl), and substituted or unsubstituted C2-20-heterocycloalkyl (e.g. C8-20-heterocycloalkyl). Preferably, R13 is selected from H, substituted or unsubstituted Ci-70-alkyl, and substituted or unsubstituted C2-7o-alkenyl, substituted or unsubstituted Ci-70-heteroalkyl, more preferably H, substituted or unsubstituted Ci-50-alkyl, substituted or unsubstituted C2-5o-alkenyl, and substituted or unsubstituted Ci-so-heteroalkyl, even more preferably substituted or unsubstituted Ci-3o-alkyl, substituted or unsubstituted C2-30-alkenyl, and substituted or unsubstituted Ci-3o-heteroalkyl, even more preferably H, substituted or unsubstituted Ci-2o-alkyl (e.g. Cs-2o-alkyl), substituted or unsubstituted C2-20-alkenyl (e.g. Cs-2o-alkenyl), and substituted or unsubstituted Ci-20-heteroalkyl (e.g. Cs-20-heteroalkyl). More preferably, R13 is selected from H, substituted or unsubstituted Ci-70-alkyl, substituted or unsubstituted C2-7o-alkenyl, and substituted or unsubstituted C6-7o-aryl, more preferably H, substituted or unsubstituted Ci-50-alkyl, and substituted or unsubstituted C2-50-alkenyl, even more preferably H, substituted or unsubstituted Ci-30-alkyl, and substituted or unsubstituted C2-3o-alkenyl, even more preferably substituted or unsubstituted Ci-2o-alkyl (e.g. Cs-20-alkyl), and substituted or unsubstituted C2-2o-alkenyl (e.g. Cs-2o-alkenyl). More preferably, R13 is selected from H, a substituted or unsubstituted Ci-70-alkyl, more preferably substituted or unsubstituted Ci-50-alkyl, even more preferably H, substituted or unsubstituted Ci-30-alkyl, even more preferably substituted or unsubstituted C1-20-alkyl (e.g. Cs-2o-alkyl). Even more preferably, R¹³ is selected from H, methyl, ethyl, n-propyl, iso-propyl, n-butyl, sec-butyl, tertbutyl, pentyl, hexyl, heptyl, octyl, phenyl, tolyl, xylyl, and methoxyphenyl. Most preferably, R13 is selected from methyl, ethyl, and n-propyl.

It may be preferred that R¹³ is not a terminal CH2 group having a sp² hybridised carbon atom. It may be preferred that Rg is not a terminal CH2 having a sp² hybridised carbon atom. It may be preferred that Rg and R13 are not terminal CH2 groups having sp² hybridised carbon atoms.

In the ester-containing substrate of Formula (I), R^{v} is an organic group having 1-70 carbon atoms. In preferred processes of the presently disclosed subject matter, R^{v} is selected from substituted or unsubstituted Ci-70-alkyl, preferably substituted or unsubstituted Ci-50-alkyl, more preferably substituted or unsubstituted Ci-30-alkyl, even more preferably substituted or unsubstituted Ci-2o-alkyl (e.g. C2-2o-alkyl or C3-2o-alkyl). Preferably, R^{v} is selected from substituted or unsubstituted C2-7o-alkyl, more preferably substituted or unsubstituted C2-5o-alkyl, even more preferably substituted or unsubstituted C2-3o-alkyl, even more preferably substituted or unsubstituted Ci-20-alkyl, C2-20-alkyl, or C3-2o-alkyl. More preferably, R^{v} is selected from methyl, ethyl, n-propyl, iso-propyl, n-butyl, sec-butyl, iso-butyl, tert-butyl, pentyl, hexyl, heptyl, and octyl. More preferably still, R^{v} is selected from methyl, ethyl, n-propyl, or iso-propyl. Even more preferably, R^{v} is ethyl, n-butyl, or tert-butyl. Most preferably, R^{v} is ethyl.

In preferred processes of the presently disclosed subject matter, the ester-containing substrate of Formula (I) is an alkyl sorbate. Preferably, the alkyl sorbate is methyl sorbate, ethyl sorbate, n-propyl sorbate, isopropyl sorbate, n-butyl sorbate, sec-butyl sorbate, or tert-butyl sorbate. More preferably, the alkyl sorbate is methyl sorbate or ethyl sorbate.

In preferred processes of the presently disclosed subject matter, the ester-containing substrate of Formula (I) is an alkyl trans-3-hexanoate. Preferably, the alkyl trans-3-hexanoate is methyl trans-3-hexanoate, ethyl trans-3-hexanoate, n-propyl trans-3-hexanoate, iso-propyl trans-3-hexanoate, n-butyl trans-3-hexanoate, sec-butyl trans-3-hexanoate, iso-butyl trans-3-hexanoate, or tert-butyl trans-3-hexanoate. More preferably the alkyl trans-3-hexanoate is methyl trans-3-hexanoate or ethyl trans-3-hexanoate.

In preferred processes of the presently disclosed subject matter, the ester-containing substrate of Formula (I) is an alkyl cis-3-hexanoate. Preferably, the alkyl cis-3-hexanoate is methyl cis-3-hexanoate, ethyl cis-3-hexanoate, n-propyl cis-3-hexanoate, iso-propyl cis-3-hexanoate, n-butyl cis-3-hexanoate, sec-butyl cis-3-hexanoate, iso-butyl cis-3-hexanoate, or tert-butyl cis-3-hexanoate. More preferably the alkyl cis-3-hexanoate is methyl cis-3-hexanoate or ethyl cis-3-hexanoate.

In preferred process of the presently disclosed subject matter, the ester-containing substrate of Formula (I) is an alkyl sorbate, an alkyl trans-3-hexanoate, or an alkyl cis-3-hexanoate as defined hereinabove.

### Alcohol Products

When the ester-containing substrate of Formula (I) is a monoester, the products of the process of the presently disclosed subject matter is an alcohol of Formula (II). In preferred processes of the presently disclosed subject matter, the conformational geometry of the alkenyl groups in the group R^{u} are the same in the ester-containing substrate of Formula(l) and the product alcohol of Formula (II).

In preferred processes of the presently disclosed subject matter the alcohol of Formula (II) is trans, trans hexa-2,4-diene-1-ol:

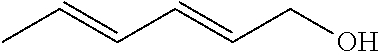

Accordingly, in preferred embodiments of the presently disclosed subject matter, the process is a process for the hydrogenation of an ester-containing substrate of Formula (I) to produce an alcohol of Formula (II) which is trans, trans hexa-2,4-diene-1-ol.

In a most preferred processes of the presently disclosed subject matter the ester-containing substrate of Formula (I) is an alkyl sorbate as defined hereinabove, and the alcohol of Formula (II) is trans, trans hexa-2,4-diene-1-ol.

In preferred processes of the presently disclosed subject matter the alcohol of Formula (II) is trans-hexa-3-ene-1-ol:

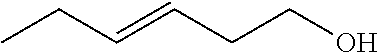

Accordingly, in preferred embodiments of the presently disclosed subject matter, the process is a process for the hydrogenation of an ester-containing substrate of Formula (I) to produce an alcohol of Formula (II) which is which is trans-hexa-3-ene-1-ol.

In a most preferred processes of the presently disclosed subject matter the ester-containing substrate of Formula (I) is an alkyl trans-3-hexanoate as defined hereinabove, and the alcohol of Formula (II) is trans-hexa-3-ene-1-ol.

In preferred processes of the presently disclosed subject matter the alcohol of Formula (II) is cis-hexa-3-ene-1-ol:

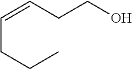

Accordingly, in preferred embodiments of the presently disclosed subject matter, the process is a process for the hydrogenation of an ester-containing substrate of Formula (I) to produce an alcohol of Formula (II) which is which is cis-hexa-3-ene-1-ol.

In a most preferred processes of the presently disclosed subject matter the ester-containing substrate of Formula (I) is an alkyl cis-3-hexanoate as defined hereinabove, and the alcohol of Formula (II) is cis-hexa-3-ene-1-ol.

### Base

The base of the presently disclosed subject matter is one whose conjugate acid has a pKa of from 4 to 15. Without wishing to be bound by any sort of theory, it is believed that the use of a base whose conjugate acid has a pKa of from 4 to 15 minimises or prevents the formation of enolate intermediates in the hydrogenation process of the presently disclosed subject matter. It is speculated that the formation of such enolate intermediates may be responsible for the reduction of the alkenyl functional group(s) and loss of the alkene regiochemistry. Furthermore, it is believed that the formation of enolate intermediates may give rise to the formation of other undesirable by-products such as those resulting from cycloaddition reactions (e.g. Diels-Alder) or condensation reactions. Moreover, it is believed that the base plays a role in the activation of the transition metal catalyst. It is therefore surprising that the base, whose conjugate acid has a pKa of from 4 to 15, functions to both activate the transition metal catalyst of the presently disclosed subject matter whilst minimising or preventing formation of enolate intermediates.

In preferred processes of the presently disclosed subject matter the conjugate acid of the base has a pKa of greater than 5, greater than 7, or greater than 9. In preferred processes of the presently disclosed subject matter the conjugate acid of the base has a pKa of less than 14, less than 13, or less than 12. In preferred processes of the presently disclosed subject matter the conjugate acid of the base has a pKa of from 5 to 14, from 7 to 13, or from 9 to 12, for example 9 to 11, for example about 10.

In preferred processes of the presently disclosed subject matter, the base is a metal phosphate or a metal carbonate wherein the conjugate acid of the base has a pKa of from 4 to 15. The metal carbonate or the metal phosphate is preferably an alkali metal phosphate, an alkali earth metal phosphate, an alkali metal carbonate, or an alkali earth metal carbonate wherein the conjugate acid of the base has a pKa of from 4 to 15.

In more preferred processes of the presently disclosed subject matter, the base is a metal phosphate wherein the conjugate acid of the base has a pKa of from 4 to 15. The metal phosphate is preferably an alkali metal phosphate or an alkali earth metal phosphate wherein the conjugate acid of the base has a pKa of from 4 to 15. The metal phosphate is more preferably an alkali metal phosphate. The alkali metal phosphate is preferably lithium phosphate (U3PO4), sodium phosphate (NasPC), potassium phosphate (K3PO4), or caesium phosphate (CS3PO4). Most preferably, the base is an alkali metal phosphate which is potassium phosphate (K3PO4).

In preferred processes of the presently disclosed subject matter, the base is present in solid form.

In preferred processes of the presently disclosed subject matter, the base is present in at least 30 mol % based upon the total amount of ester-containing substrate, preferably in at least 35 mol % based upon the total amount of ester-containing substrate, more preferably in at least 40 mol % based upon the total amount of ester-containing substrate, and even more preferably in at least 45 mol % based upon the total amount of ester-containing substrate.

In preferred processes of the presently disclosed subject matter, the base is present in less than or equal to 200 mol % based upon the total amount of ester-containing substrate, more preferably in less than or equal to 125 mol % based upon the total amount of ester-containing substrate.

In preferred processes of the presently disclosed subject matter, the base is present in a range of 30 to 70 mol % based upon the total amount of ester-containing substrate, more preferably in a range of 30 to 60 mol % based upon the total amount of ester-containing substrate, even more preferably in a range of 30 to 50 mol % based upon the total amount of ester-containing substrate.

In preferred processes of the presently disclosed subject matter the base is present in the process as a solid. In preferred processes of the presently disclosed subject matter the base is substantially insoluble in the ester-containing substrate of Formula (I) and/or the alcohol of Formula (II). In preferred processes of the presently disclosed subject matter the base is substantially insoluble in the solvent.

In preferred processes of the presently disclosed subject matter, the process further comprises the step of separating the base by filtration. The step of filtering the base after filtration is carried out after the hydrogenation of the ester-containing substrate of Formula (I).

Using a base which is substantially insoluble in the ester-containing substrate of Formula (I) and/or the alcohol of Formula (II) and/or the solvent has the advantage that is can be easily separated from the reaction products, for example from the alcohol of Formula (II).

Bases, such as potassium phosphate (K3PO4) which are insoluble in organic solvents, such as toluene and THF, therefore offer additional advantages due to their ease of separation by, for example, filtration.

### Solvent

In preferred processes of the presently disclosed subject matter, the process is carried out in the absence of solvent. This has the advantage of making the process both easier and less expensive to perform.

In alternative preferred processes of the presently disclosed subject matter, the process is carried out in the presence of a solvent.

Preferably, the solvent is selected from an alcohol, toluene, THF and Me-THF. More preferably, the solvent is selected from toluene, THF and Me-THF. Most preferably, the solvent is selected from toluene and THF.

In preferred processes of the presently disclosed subject matter, the solvent is present in an amount of 10 to 100 vol % based upon the total volume of the ester-containing substrate, preferably 15 to 95 vol % based upon the total volume of the ester-containing substrate, more preferably 20 to 90 vol % based upon the total volume of the ester-containing substrate (e.g. 50 vol % based upon the total volume of the ester-containing substrate).

In preferred processes of the presently disclosed subject matter, the volume ratio of the solvent to the ester-containing substrate is less than or equal to 1:1, preferably less than or equal to 1:2.

In preferred processes of the presently disclosed subject matter, the volume ratio of the solvent to the ester-containing substrate is in the range 1:2 to 1:1, preferably in the range 1:2 to 1:1.5.

In preferred processes of the presently disclosed subject matter, the process is carried out in the presence of more than one solvent. Preferred solvents are as described above.

In alternative preferred processes of the presently disclosed subject matter, the process is carried out in the presence of a first solvent and a second solvent.

In preferred processes of the presently disclosed subject matter, the first solvent is selected from toluene, THF and Me-THF. In preferred processes of the presently disclosed subject matter, the second solvent is an alcohol, preferably methanol or ethanol.

In particularly preferred processes of the presently disclosed subject matter, the first solvent is toluene and the second solvent is an alcohol, preferably methanol or ethanol.

In alternative particularly preferred processes of the presently disclosed subject matter, the first solvent is THE and the second solvent is an alcohol, preferably methanol or ethanol.

In preferred processes of the presently disclosed subject matter, the first solvent is present in an amount of 10 to 100 vol % based upon the total volume of the ester-containing substrate, preferably 15 to 95 vol % based upon the total volume of the ester-containing substrate, more preferably 20 to 90 vol % based upon the total volume of the ester-containing substrate (e.g. 50 vol % based upon the total volume of the ester-containing substrate).

In preferred processes of the presently disclosed subject matter, the volume ratio of the first solvent to the ester-containing substrate is less than or equal to 1:1, preferably less than or equal to 1:2.

In preferred processes of the presently disclosed subject matter, the volume ratio of the first solvent to the ester-containing substrate is in the range 1:2 to 1:1, preferably in the range 1:2 to 1:1.5.

In preferred processes of the presently disclosed subject matter, the second solvent is present in an amount of 1 to 15 vol % based upon the total volume of ester-containing substrate, preferably in an amount of 1 to 10 vol % based upon the total volume of ester-containing substrate, preferably in an amount of 1 to 7.5 vol % based upon the total volume of the ester-containing substrate, more preferably in an amount of 1 to 5 vol % based upon the total volume of the ester-containing substrate.

In preferred processes of the presently disclosed subject matter, the first solvent is present in an amount of 10 to 100 vol % based upon the total volume of the ester-containing substrate and the second solvent is present in an amount of 1 to 10 vol % based upon the total volume of ester-containing substrate, preferably the first solvent is present in an amount of 15 to 95 vol % based upon the total volume of the ester-containing substrate and the second solvent is present in an amount of 1 to 7.5 vol % based upon the total volume of the ester-containing substrate, more preferably the first solvent is present in an amount of 20 to 90 vol % based upon the total volume of the ester-containing substrate and the second solvent is present in an amount of 1 to 5 vol % based upon the total volume of the ester-containing substrate.

### By-Products

The process of the presently disclosed subject matter has the surprising advantage of producing lower levels of unwanted by-products.

As described hereinabove, in processes of the prior art and/or where a strong base (e.g. one whose conjugate acid has a pKa of greater than 15, such a metal alkoxide) is used unwanted by-products may be formed.

Unwanted by-products typically comprise wax esters, saturated alcohols, saturated esters, and hemiacetals. Without wishing to be bound by any sort of theory, the formation of these unwanted by-products are believed to result from the formation of enolate intermediate compounds. Accordingly, in the process of the presently disclosed subject matter it is believed that lower levels of unwanted by-products are formed because the formation of enolate intermediates is minimised or eliminated.

### Temperature

The process of the presently disclosed subject matter may be conducted at a temperature in the range 20 to 150° C. In preferred processes of the presently disclosed subject matter the process may be conducted at a temperature in the range 40 to 90° C., more preferably in the range 40 to 85° C., even more preferably in the range 50 to 85° C., even more preferably in the range 50 to 80° C., even more preferably in the range 55 to 75° C., and most preferably in the range 60 to 75° C. (e.g. about 70° C.).

The preferred processes of the presently disclosed subject matter are conducted at relatively low temperatures. It has surprisingly been found that using a temperature within the preferred ranges described hereinabove produces fewer undesirable by-products than when higher temperatures are used whilst maintaining a good rate of reaction.

In particularly preferred embodiments of the process the solvent is toluene and the process is conducted at a temperature in the range 40 to 90° C., more preferably in the range 45 to 85° C., even more preferably in the range 50 to 85° C., even more preferably in the range 50 to 80° C., even more preferably in the range 55 to 75° C., and most preferably in the range 60 to 75° C. (e.g. about 70° C.).

It is surprising that when toluene is the solvent that temperatures lower than the atmospheric pressure boiling point of toluene (ca. 110° C.) gives improved uptake of hydrogen gas in the hydrogenation process.

### Pressure

Preferred processes of the presently disclosed subject matter are conducted at a pressure that is at least 5 bar, more preferably at least 10 bar, even more preferably at least 20 bar, even more preferably at least 30 bar, even more preferably at least 40 bar, and most preferably at least 50 bar.

Preferred processes of the presently disclosed subject matter are conducted at a pressure that is in the range 5 to 100 bar, more preferably in the range 10 to 95 bar, even more preferably in the range 20 to 90 bar, even more preferably in the range 25 to 70 bar, and most preferably in the range 30 to 50 bar.

### Duration

Preferred processes of the presently disclosed subject matter are conducted for a duration of 1 to 24 hours, more preferably 2 to 20 hours, even more preferably 3 to 18 hours, and most preferably 4 to 16 hours.

### S/C—Substrate to Catalyst

In preferred processes of the presently disclosed subject matter, the substrate/catalyst loading is greater than or equal to 500/1, preferably greater than or equal to 650/1, even more preferably greater than or equal to 750/1, even more preferably greater than or equal to 850/1. In preferred processes of the presently disclosed subject matter, the substrate/catalyst loading is less than or equal to 50,000/1, preferably less than or equal to 30,000/1, even more preferably less than or equal to 20,000/1, even more preferably less than or equal to 10,000/1. For example, in preferred processes of the presently disclosed subject matter, the substrate/catalyst loading is greater than or equal to 500/1 and less than or equal to 50,000/1, preferably the substrate/catalyst loading is greater than or equal to 650/1 and less than or equal to 30,000/1, even more preferably the substrate/catalyst loading is greater than or equal to 750/1 and less than or equal to 20,000/1, even more preferably the substrate/catalyst loading is greater than or equal to 1,000/1 and less than or equal to 10,000/1, for example 5,000/1.

### Transition Metal Catalyst

The process of the presently disclosed subject matter employs a transition metal catalyst. The transition metal catalyst may be pre-formed or may be formed in situ during the ester hydrogenation reaction. Preferably, the transition metal catalyst is pre-formed. Alternatively, the transition metal catalyst is formed in situ during the ester hydrogenation reaction.

In preferred processes of the presently disclosed subject matter, the transition metal in the transition metal catalyst is a Group 6, Group 7, Group 8, or Group 9 transition metal. More preferably, the transition metal in the transition metal catalyst is a Group 7, Group 8, or Group 9 transition metal. Even more preferably, the transition metal in the transition metal catalyst is a Group 8 transition metal.

In preferred processes of the presently disclosed subject matter, the transition metal in the transition metal catalyst is selected from Mo, Mn, Fe, Ru, Co and Os. More preferably, the transition metal in the transition metal catalyst is selected from Ru and Os. Most preferably, the transition metal in the transition metal catalyst is Ru.

In preferred processes of the presently disclosed subject matter, the transition metal catalyst employed in the process of the presently disclosed subject matter comprises a tridentate ligand.

In preferred processes of the presently disclosed subject matter, the transition metal catalyst comprises a tridentate ligand having a Formula (III)

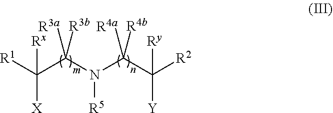

wherein:
X is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R¹ and R^{x} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl, or R¹ and one of R^{3a} and R^{3b} or R^{x} and one of R^{3a} and R^{3b} together with the atoms to which they are bound, form a ring;
or X is a heteroatom and when taken together with R¹ it forms an optionally substituted heterocycle when R^{x} is absent;
Y is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R² and R^{y} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl, or R² and one of R^{4a} and R^{4b} or R^{y} and one of R^{4a} and R^{4b} together with the atoms to which they are bound, form a ring;
or Y is a heteroatom and when taken together with R² it forms an optionally substituted heterocycle when R^{y} is absent;
R^{3a}, R^{3b}, R^{4a}, and R^{4b} are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl, or R^{3a} and one of R^{4a} and R^{4b} or R^{3b} and one of R^{4a} and R^{4b} together with the atoms to which they are bound, form a heterocycle;
R⁵ is selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl;
each m and n is independently 1 or 2; and
R^{a} and R^{b}, if present, are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; or when X and/or Y is —NR^{a}R^{b}, —PR^{a}R^{b}, —OPR^{a}R^{b}, or —NHPR^{a}R^{b}, R^{a} and R^{b} together with the heteroatom to which they are attached form a heterocycle.

In tridentate ligands of Formula (III), X is preferably selected from —SR^{a}, —CR^{a}, —NR^{a}R^{b}, —PR^{a}R^{b}, and —NHPR^{a}R^{b}. More preferably, X is selected from —SR^{a}, —PR^{a}R^{b}, and —NHPR^{a}R^{b}. Even more preferably, X is selected from —SR^{a} and —PR^{a}R^{b}. Most preferably, X is —SR^{a}.

In tridentate ligands of Formula (III), R¹ and R^{x} are each independently preferably selected from hydrogen, substituted or unsubstituted Ci-20-alkyl, substituted or unsubstituted C1·20-heteroalkyl and substituted or unsubstituted C3-2o-cycloalkyl. More preferably, R¹ and R^{x} are each independently selected from hydrogen and substituted or unsubstituted Ci-2o-alkyl. Even more preferably, R¹ and R^{x} are each hydrogen.

In alternative preferred tridentate ligands of Formula (III) R^{x} is absent, X is a heteroatom and when taken together with R¹ it forms an optionally substituted heterocycle. More preferably, R^{x} is absent, X is a heteroatom and when taken together with R¹ it forms an optionally substituted heteroaromatic ring. More preferably, the optionally substituted heteroaromatic ring is an optionally substituted nitrogen-containing heteroaromatic ring. Even more preferably, the optionally substituted nitrogen-containing heteroaromatic ring is selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl. Even more preferably, the optionally substituted nitrogen-containing heteroaromatic ring is selected from pyridinyl, pyrrolyl, imidazolyl, pyrazolyl, triazolyl, tetrazolyl, and pyrimidyl. Most preferably, the optionally substituted nitrogen-containing heteroaromatic ring is pyridinyl.

In tridentate ligands of Formula (III), Y is preferably selected from —SR^{a}, —CR^{a}, —NR^{a}R^{b}, —PR^{a}R^{b}, and —NHPR^{a}R^{b}. More preferably, Y is selected from —SR^{a}, —PR^{a}R^{b}, and —NHPR^{a}R^{b}. Even more preferably, Y is selected from —SR^{a} and —PR^{a}R^{b}. Most preferably, Y is —SR. In tridentate ligands of Formula (III), R² and R^{y} are each independently preferably selected from hydrogen, substituted or unsubstituted Ci-20-alkyl, substituted or unsubstituted C1·20-heteroalkyl and substituted or unsubstituted C3-2o-cycloalkyl. More preferably, R² and R^{y} are each independently selected from hydrogen and substituted or unsubstituted Ci-2o-alkyl. Even more preferably, R² and R^{y} are each hydrogen.

In alternative preferred tridentate ligands of Formula (III), Y is a heteroatom and when taken together with R² it forms an optionally substituted heterocycle when R^{y} is absent. More preferably, Y is a heteroatom and when taken together with R² it forms an optionally substituted heteroaromatic ring when R^{y} is absent. More preferably, the optionally substituted heteroaromatic ring is an optionally substituted nitrogen-containing heteroaromatic ring. Even more preferably, the optionally substituted nitrogen-containing heteroaromatic ring is selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl. Even more preferably, the optionally substituted nitrogen-containing heteroaromatic ring is selected from pyridinyl, pyrrolyl, imidazolyl, pyrazolyl, triazolyl, tetrazolyl, and pyrimidyl. Most preferably, the optionally substituted nitrogen-containing heteroaromatic ring is pyridinyl.

In tridentate ligands of Formula (III), R^{3a}, R^{3b}, R^{4a}, and R^{4b} are each independently preferably selected from hydrogen, substituted or unsubstituted Ci-20-alkyl, substituted or unsubstituted Ci-2o-heteroalkyl and substituted or unsubstituted C3-2o-cycloalkyl. More preferably, R^{3a}, R^{3b}, R^{4a}, and R^{4b} are each independently selected from hydrogen and substituted or unsubstituted Ci-2o-alkyl. Even more preferably, R^{3a}, R^{3b}, R^{4a}, and R^{4b} are each hydrogen.

In alternative preferred tridentate ligands of Formula (III), R^{3a} and one of R^{4a} and R^{4b} or R^{3b} and one of R^{4a} and R^{4b} together with the atoms to which they are bound, form a heterocycle. Preferably, the heterocycle is a six-membered ring heterocycle.

In tridentate ligands of Formula (III), R⁵ is preferably selected from hydrogen, substituted or unsubstituted Ci-20-alkyl, substituted or unsubstituted Ci-20-heteroalkyl and substituted or unsubstituted C3-2o-cycloalkyl. More preferably, R⁵ is selected from hydrogen and substituted or unsubstituted Ci-2o-alkyl. Even more preferably, R⁵ is hydrogen.

In tridentate ligands of Formula (III), each m and n is preferably 1.

In tridentate ligands of Formula (III), R^{a} and R^{b}, if present, are each independently preferably selected from hydrogen, substituted or unsubstituted Ci-20-alkyl, substituted or unsubstituted Ci-2o-heteroalkyl, substituted or unsubstituted C3-2o-cycloalkyl, substituted or unsubstituted Ce-20-aryl, and substituted or unsubstituted C4-2o-heteroaryl. More preferably, R^{a} and R^{b}, if present, are each independently selected from hydrogen, substituted or unsubstituted C1·20-alkyl (e.g. Ci-10-alkyl) and substituted or unsubstituted C6-2o-aryl. Particularly preferred C1·20-alkyl groups include ethyl, ethyl, n-propyl, iso-propyl, n-butyl, sec-butyl, tert-butyl, pentyl, and hexyl, more preferably methyl, ethyl, iso-propyl, tert-butyl, even more preferably ethyl. Preferred C6-2o-aryl groups include phenyl, tolyl, xylyl, and methoxyphenyl, more preferably phenyl.

In alternative preferred tridentate ligands of Formula (III), when X and/or Y is —NR^{a}R^{b}, —PR^{a}R^{b}, —OPR^{a}R^{b}, or —NHPR^{a}R^{b}, R^{a} and R^{b} together with the heteroatom to which they are attached form a heterocycle.

In preferred processes of the presently disclosed subject matter, the transition metal catalyst comprises a tridentate ligand having a Formula (III)
wherein:
X is selected from —SR^{a}, —CR^{a}, —NR^{a}R^{b}, —PR^{a}R^{b}, and —NHPR^{a}R^{b};
R¹ and R^{x} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or X is a heteroatom and when taken together with R¹ it forms an optionally substituted heteroaromatic ring when R^{x} is absent, wherein the heteroaromatic ring is a nitrogen containing heteroaromatic ring;
Y is selected from —SR^{a}, —CR^{a}, —NR^{a}R^{b}, —PR^{a}R^{b}, and —NHPR^{a}R^{b};
R² and R^{y} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or Y is a heteroatom and when taken together with R² it forms an optionally substituted heteroaromatic ring when R^{y} is absent, wherein the heteroaromatic ring is a nitrogen containing heteroaromatic ring;
R^{3a}, R^{3b}, R^{4a}, R^{4b} and R⁵ are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o -heteroaryl; each m and n is independently 1 or 2; and
R^{a} and R^{b}, if present, are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; or when X and/or Y is —NR^{a}R^{b}, —PR^{a}R^{b} or —NHPR^{a}R^{b}, R^{a} and R^{b} together with the heteroatom to which they are attached form a heterocycle.

In preferred processes of the presently disclosed subject matter, the transition metal catalyst comprises a tridentate ligand having a Formula (III)
wherein:
X is selected from —SR^{a}, —PR^{a}R^{b}, and —NHPR^{a}R^{b};
R¹ and R^{x} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or X is a heteroatom and when taken together with R¹ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
Y is selected from —SR^{a}, —PR^{a}R^{b}, and —NHPR^{a}R^{b};
R² and R^{y} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or Y is a heteroatom and when taken together with R² it forms an optionally substituted heteroaromatic ring when R^{y} is absent, wherein the heteroaromatic ring is a nitrogen containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl; R^{3a}, R^{3b} R^{4a}, R^{4b} and R⁵ are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted C1-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; each m and n is independently 1 or 2; and
R^{a} and R^{b}, if present, are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; or when X and/or Y is —PR^{a}R^{b} or —NHPR^{a}R^{b}, R^{a} and R^{b} together with the heteroatom to which they are attached form a heterocycle.

In preferred processes of the presently disclosed subject matter, the transition metal catalyst comprises a tridentate ligand having a Formula (III)
wherein:
X is selected from —SR^{a} and —PR^{a}R^{b};
R¹ and R^{x} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or X is a heteroatom and when taken together with R¹ it forms an optionally substituted heteroaromatic ring when R^{x} is absent, wherein the heteroaromatic ring is a nitrogen containing heteroaromatic ring selected from pyridinyl, pyrrolyl, imidazolyl, pyrazolyl, triazolyl, tetrazolyl, and pyrimidyl;
Y is selected from —SR^{a} and —PR^{a}R^{b};
R² and R^{y} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or Y is a heteroatom and when taken together with R² it forms an optionally substituted heteroaromatic ring when R^{y} is absent, wherein the heteroaromatic ring is a nitrogen containing heteroaromatic ring selected from pyridinyl, pyrrolyl, imidazolyl, pyrazolyl, triazolyl, tetrazolyl, and pyrimidyl;
R^{3a}, R^{3b} R^{4a}, R^{4b} and R⁵ are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; each m and n is independently 1 or 2; and
R^{a} and R^{b}, if present, are each independently selected from hydrogen, substituted or unsubstituted Ci-20-alkyl, substituted or unsubstituted C2-2o-alkenyl, substituted or unsubstituted C2-2o-alkynyl, substituted or unsubstituted C1-20-heteroalkyl, substituted or unsubstituted Ci-20-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; or when X and/or Y is —PR^{a}R^{b}, R^{a} and R^{b} together with the heteroatom to which they are attached form a heterocycle.

In preferred processes of the presently disclosed subject matter, the transition metal catalyst comprises a tridentate ligand having a Formula (III), wherein:
X is —SR^{a};
R¹ and R^{x} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-20-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
Y is —SR^{a};
R² and R^{y} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
R^{3a}, R^{3b}, R^{4a}, R^{4b} and R⁵ are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted C1-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; each m and n is independently 1 or 2; and
R^{a} are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl.

Preferably, the transition metal catalyst comprises a tridentate ligand having a Formula (III),
wherein:
X is —SR^{a};
R¹ and R^{x} are each independently selected from hydrogen, substituted or unsubstituted C1·20-alkyl, substituted or unsubstituted Ci-20-heteroalkyl, and substituted or unsubstituted C3-20-cycloalkyl;
Y is —SR^{a};
R² and R^{y} are each independently selected from hydrogen, substituted or unsubstituted C1·20-alkyl, substituted or unsubstituted Ci-20-heteroalkyl, and substituted or unsubstituted C3-20-cycloalkyl;
R^{3a}, R^{3b} R^{4a}, R^{4b} and R⁵ are each independently selected from hydrogen, substituted or unsubstituted Ci-20-alkyl, substituted or unsubstituted Ci-20-heteroalkyl, and substituted or unsubstituted C3-2o-cycloalkyl;
each m and n is independently 1 or 2; and
R^{a} are each independently selected from hydrogen, substituted or unsubstituted Ci-20-alkyl, substituted or unsubstituted Ci-20-heteroalkyl, and substituted or unsubstituted C3-2o-cycloalkyl.

More preferably, the transition metal catalyst comprises a tridentate ligand having a Formula (III), wherein:
X is —SR^{a};
R¹ and R^{x} are each independently selected from hydrogen and substituted or unsubstituted Ci-2o-alkyl;
Y is —SR^{a};
R² and R^{y} are each independently selected from hydrogen and substituted or unsubstituted C1·0-alkyl;
R^{3a}, R^{3b} R^{4a}, R^{4b} and R⁵ are each independently selected from hydrogen and substituted or unsubstituted Ci-2o-alkyl;
each m and n is independently 1 or 2; and
R^{a} are each independently selected from hydrogen and substituted or unsubstituted C1·20-alkyl, substituted or unsubstituted Ci-20-heteroalkyl, and substituted or unsubstituted C3-20-cycloalkyl.

Even more preferably, the transition metal catalyst comprises a tridentate ligand having a Formula (III), wherein:
X and Y are each —SR^{a};
R¹, R^{x}, R², R^{y}, R^{3a}, R^{3b}, R^{4a}, R^{4b} and R⁵ are each hydrogen;
m and n are each 1; and
R^{a} are each independently substituted or unsubstituted Ci-20-alkyl, preferably C1·10 alkyl.

Even more preferably, the transition metal catalyst comprises a tridentate ligand having a Formula (III), wherein:
X and Y are each —SEt;
R¹, R^{x}, R², R^{y}, R^{3a}, R^{3b}, R^{4a}, R^{4b} and R⁵ are each hydrogen; and
m and n are each 1.

In alternative preferred processes of the presently disclosed subject matter, the transition metal catalyst comprises a tridentate ligand having a Formula (III), wherein:
X is a heteroatom and when taken together with R¹ it forms an optionally substituted heteroaromatic ring when R^{x} is absent;
Y is —PR^{a}R^{b};
R² and R^{y} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
R^{3a}, R^{3b} R^{4a}, R^{4b} and R⁵ are each independently selected from hydrogen, substituted or unsubstituted Ci-20-alkyl, substituted or unsubstituted C2-20-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; each m and n is independently 1 or 2; and
R^{a} and R^{b} are each independently selected from hydrogen, substituted or unsubstituted C1·20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl; or R^{a} and R^{b} together with the heteroatom to which they are attached form a heterocycle.

Preferably, the transition metal catalyst comprises a tridentate ligand having a Formula (III), wherein:
X is a nitrogen atom and when taken together with R¹ it forms an optionally substituted heteroaromatic ring when R^{x} is absent, wherein the heteroaromatic ring is a nitrogen containing heteroaromatic ring;
Y is —PR^{a}R^{b};
R² and R^{y} are each independently is selected from hydrogen, substituted or unsubstituted Ci-20-alkyl, substituted or unsubstituted Ci-20-heteroalkyl, and substituted or unsubstituted C3-20-cycloalkyl;
R^{3a}, R^{3b}, R^{4a}, R^{4b} and R⁵ are each independently selected from hydrogen, substituted or unsubstituted Ci-20-alkyl, substituted or unsubstituted Ci-20-heteroalkyl, substituted or unsubstituted C3-2o-cycloalkyl;
each m and n is independently 1 or 2; and
R^{a} and R^{b} are each independently selected from substituted or unsubstituted Ci-20-alkyl, substituted or unsubstituted Ci-20-heteroalkyl, substituted or unsubstituted C3-2o-cycloalkyl, substituted or unsubstituted C6-2o-aryl, and substituted or unsubstituted C4-2o-heteroaryl.

More preferably, the transition metal catalyst comprises a tridentate ligand having a Formula (III), wherein:
X is a nitrogen atom and when taken together with R¹ it forms an optionally substituted heteroaromatic ring when R^{x} is absent, wherein the heteroaromatic ring is a nitrogen containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
Y is —PR^{a}R^{b};
R² R^{y}, R^{3a}, R^{3b}, R^{4a}, R^{4b} and R⁵ are each hydrogen;
each m and n is 1; and
R^{a} and R^{b} are each independently selected from substituted or unsubstituted Ci-2o-alkyl and substituted or unsubstituted C6-2o-aryl.

Even more preferably, the transition metal catalyst comprises a tridentate ligand having a Formula (III), wherein:
X is a nitrogen atom and when taken together with R¹ it forms an optionally substituted heteroaromatic ring when R^{x} is absent, wherein the heteroaromatic ring is a nitrogen containing heteroaromatic ring selected from pyridinyl, pyrrolyl, imidazolyl, pyrazolyl, triazolyl, tetrazolyl, and pyrimidyl;
Y is —PR^{a}R^{b};
R² R^{y}, R^{3a}, R^{3b}, R^{4a}, R^{4b} and R⁵ are each hydrogen;
each m and n is 1; and
R^{a} and R^{b} are each independently selected from methyl, ethyl, n-propyl, iso-propyl, n-butyl, sec-butyl, tert-butyl, pentyl, hexyl, phenyl, tolyl, xylyl, and methoxyphenyl.

Even more preferably, the transition metal catalyst comprises a tridentate ligand having a Formula (III), wherein:
X is a nitrogen atom and when taken together with R¹ it forms an optionally substituted pyridinyl ring when R^{x} is absent;
Y is —PR^{a}R^{b};
R² R^{y}, R^{3a}, R^{3b}, R^{4a}, R^{4b} and R⁵ are each hydrogen;
each m and n is 1; and
R^{a} and R^{b} are each independently selected from methyl, ethyl, iso-propyl, tert-butyl, phenyl, tolyl, xylyl, and methoxyphenyl.

Even more preferably, the transition metal catalyst comprises a tridentate ligand having a Formula (III), wherein:
X is a nitrogen atom and when taken together with R¹ it forms an optionally substituted pyridinyl ring when R^{x} is absent;
Y is —PR^{a}R^{b};
R² R^{y}, R^{3a}, R^{3b}, R^{4a}, R^{4b} and R⁵ are each hydrogen;
each m and n is 1; and
R^{a} and R^{b} are each phenyl.

In preferred processes of the presently disclosed subject matter, the transition metal catalyst has a Formula (IV) or Formula (V)

[M(L¹)(L²)_{d}] (IV)

[M(L¹)(L²)_{d}]W (V)
wherein:
M is a transition metal;
L¹ is a tridentate ligand as hereinbefore defined;
L² are ligands which may be the same or different;
d is 1, 2 or 3; and
W is a non-coordinated anionic ligand.

In preferred processes of the presently disclosed subject matter, M is a Group 6, Group 7, Group 8, or Group 9 transition metal. More preferably, M is a Group 7, Group 8, or Group 9 transition metal. Even more preferably, M is a Group 8 transition metal.

In preferred processes of the presently disclosed subject matter, M is a transition metal selected from Mo, Mn, Fe, Ru, Co and Os. More preferably, M is a transition metal selected from Ru and Os. Most preferably, M is Ru.

In preferred processes of the presently disclosed subject matter, d is 3.

As will be understood by a skilled person, each L² may be a monodentate ligand or a multidentate ligand, provided the combination of L² ligands is allowed by the rules of valency. In preferred processes of the presently disclosed subject matter, each L² is a monodentate ligand. Preferably, each L² is independently a neutral monodentate ligand or an anionic monodentate ligand. In preferred processes of the presently disclosed subject matter, each L² is independently selected from —H, —CO, —CN, —P(R′)s, —As(R′)s, —CR′, —OR′, —O(C═O)R′, —NR′2, halogen (e.g. —Cl, —Br, —I), and solvent, wherein each R′ is independently selected from substituted or unsubstituted alkyl, substituted or unsubstituted alkenyl, substituted or unsubstituted alkynyl, substituted or unsubstituted heteroalkyl, substituted or unsubstituted alkoxy, substituted or unsubstituted cycloalkyl, substituted or unsubstituted heterocycloalkyl, substituted or unsubstituted aryl, and substituted or unsubstituted heteroaryl. Preferably, each L² is independently selected from —H, —CO, —P(R′)s, and halogen. More preferably, each L² is independently selected from —CO, —PPhs, and —Cl. When L² is solvent, the solvent is preferably selected from THF, Me-THF, MeCN, H2O and an alcohol (e.g. methanol, ethanol, iso-propanol etc.).

In the transition metal catalysts of Formula (V), W is a non-coordinated anionic ligand. By “non-coordinated anion ligand”, we mean the anionic ligand is forced to the outer sphere of the metal centre. The anionic ligand, therefore, is dissociated from the metal centre. This is in contrast to neutral complexes in which the anionic ligand is bound to the metal within the coordination sphere. The anionic ligand can be generally identified as non-coordinating by analysing the X-ray crystal structure of the cationic complex. Preferably, W is selected from the group consisting of triflate (i.e. TfO′ or CF3SOT), tetrafluoroborate (i.e. —BF4), hexafluoroantimonate (i.e. —SbFe), hexafluorophosphate (PFe′), [B[3,5-(CF3)2CeH3]4]′([BArF4]′), halide (e.g. Cl; Br, I′) and mesylate (MsO— or MeSOT).

Preferably, the transition metal catalyst is a transition metal catalyst of Formula (IV).

Alternatively, the transition metal catalyst is a transition metal catalyst of Formula (V).

In preferred processes of the presently disclosed subject matter, the transition metal catalyst is a transition metal catalyst selected from:

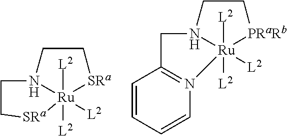

for example:

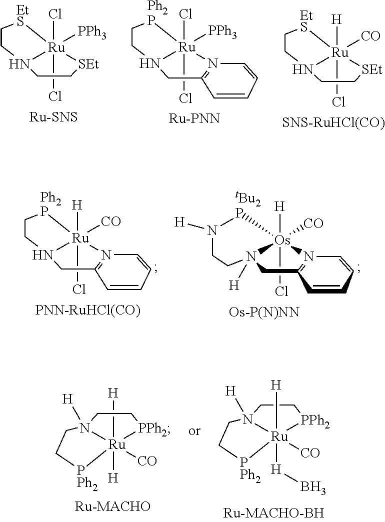

In preferred processes of the presently disclosed subject matter, the transition metal catalyst is

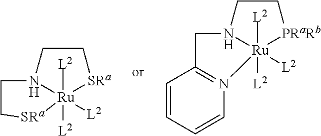

In preferred processes of the presently disclosed subject matter, the transition metal catalyst is Ru—SNS, Ru—SNN, or Ru—PNN. More preferably, the transition metal catalyst is Ru—SNS or Ru—PNN.

In preferred processes of the presently disclosed subject matter, the transition metal catalyst is

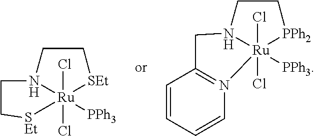

In preferred processes of the presently disclosed subject matter, the transition metal catalyst employed in the processes of the presently disclosed subject matter comprises a bidentate ligand.

In preferred processes of the presently disclosed subject matter, the transition metal catalyst comprises a bidentate ligand having a Formula (VI)

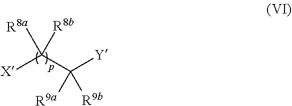

wherein:
X′ is —NHR^{ax};
Y′ is selected from —SR^{ax}, —OR^{ax}, —CR^{ax}, —NR^{ax}R^{bx}, —PR^{ax}R^{bx}, —P(═O)R^{ax}R^{bx}, —OPR^{ax}R^{bx}, and —NHPR^{ax}R^{bx};
R^{8a}, R^{8b}, R^{9a} and R^{9b} are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or
unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; p is 1 or 2; and
R^{ax} and R^{bx}, if present, are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; or when X′ and/or Y′ is —NR^{ax}R^{bx}, —PR^{ax}R^{bx}, —OPR^{ax}R^{bx}, or —NHPR^{ax}R^{bx}, R^{ax} and R^{bx} together with the heteroatom to which they are attached form a heterocycle.

In preferred processes of the presently disclosed subject matter, the transition metal catalyst has a Formula (VII) or Formula (VIII)

[M(L¹)e(L²)_{f}] (VII)

[M(L¹)ₑ(L²)_{f}]W (VIII)
wherein:
M is a transition metal;
L¹ are bidentate ligands as hereinbefore defined which may be the same or different;
L², if present, are ligands which may be the same or different;
e is 1 or 2 such that when e is 1, f is 2, 3 or 4, and when e is 2, f is 0, 1 or 2; and
W is a non-coordinated anionic ligand.
M, L² and W are as generally described above.

In preferred processes of the presently disclosed subject matter, the transition metal catalyst employed in the processes of the presently disclosed subject matter comprises a tetradentate ligand.

In preferred processes of the presently disclosed subject matter, the transition metal catalyst comprises a tetradentate ligand having a Formula (IX)

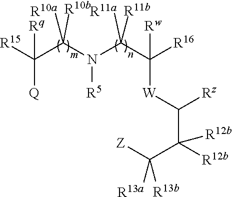

wherein:
Q is selected from —SR^{ay}, —OR^{ay}, —CR^{ay}, —NR^{ay}R^{by}, —PR^{ay}R^{by}, —P(═O)R^{ay}R^{by}, —OPR^{ay}R^{by}, and —NHPR^{ay}R^{by};
R¹⁵ and R^{q} are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or Q is a heteroatom and when taken together with R¹⁵ it forms an optionally substituted heterocycle when R^{a} is absent;
W is selected from S, O, NR^{a}, and PR^{a};
R¹⁶, R^{w} and R^{Z} are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-2o-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted Ce-2o-aryl, and substituted or unsubstituted C4-2o-heteroaryl;
or R¹⁶ when taken together with R^{z} forms an optionally substituted heterocycle when R^{w} is absent;
Z is selected from —SR^{ay}, —OR^{ay}, —CR^{ay}, —NR^{ay}R^{by}, —PR^{ay}R^{by}, —P(═O)R^{ay}R^{by}, —OPR^{ay}R^{by}, and —NHPR^{ay}R^{by};
Ri° a, R^{10b}, R^{11a}, and R^{11b} are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-2o-heteroalkyl, substituted or
unsubstituted Ci-2o-alkoxy, substituted or unsubstituted C3-2o-cycloalkyl, substituted or unsubstituted C3-20-cycloalkenyl, substituted or unsubstituted C2-20-heterocycloalkyl, substituted or unsubstituted C6-2o-aryl, and substituted or unsubstituted C4-2o-heteroaryl; or R^{10a} and one of R^{11a} and R^{11b} or R^{10b} and one of R^{11a} and R^{11b} together with the atoms to which they are bound, form a heterocycle;
Ri²³ R^{12b}, R^{13a}, R^{13b} and R¹⁴ are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; each q and r is independently 1 or 2;
s is 0, 1 or 2; and
R^{ay} and R^{by}, if present, are each independently selected from hydrogen, substituted or unsubstituted Ci-20-alkyl, substituted or unsubstituted C2-2o-alkenyl, substituted or unsubstituted C2-2o-alkynyl, substituted or unsubstituted Ci-20-heteroalkyl, substituted or unsubstituted Ci-2o-alkoxy, substituted or unsubstituted C3-2o-cycloalkyl, substituted or unsubstituted C3-2o-cycloalkenyl, substituted or unsubstituted C2-20-heterocycloalkyl, substituted or unsubstituted C6-2o-aryl, and substituted or unsubstituted C4-2o-heteroaryl; or when Q and/or Z is —NR^{ay}R^{by}, —PR^{ay}R^{by}, —OPR^{ay}R^{by}, or —NHPR^{ay}R^{by}, R^{ay} and R^{by} together with the heteroatom to which they are attached form a heterocycle. In preferred processes of the presently disclosed subject matter, the transition metal catalyst has a Formula (X) or Formula (XI)

[M(L¹)(L²)_{g}] (X)

[M(L¹)(L²)_{g}]W (XI)
wherein:
M is a transition metal;
L¹ is a tetradentate ligand as hereinbefore defined;
L², if present, are ligands which may be the same or different;
g is 0, 1 or 2; and
W is a non-coordinated anionic ligand.
   M, L² and W are as generally described above.

In preferred processes of the presently disclosed subject matter, the transition metal catalyst is removed from the reaction mixture by a precipitation step using a co-solvent.

In alternative preferred processes of the presently disclosed subject matter, the transition metal catalyst is removed from the reaction mixture by distillation of the product.

In alternative preferred processes of the presently disclosed subject matter, the transition metal catalyst is removed from the reaction mixture by crystallisation of the product.

In alternative preferred processes of the presently disclosed subject matter, the transition metal catalyst is removed from the reaction mixture using a metal scavenger.

In preferred processes of the presently disclosed subject matter, the process is a flow process. Preferably, the process is a flow process wherein any excess base is recycled or reused. The base may be recycled or reused once, twice, three times, or more.

The presently disclosed subject matter will now be further described by way of the following non-limiting examples.

### EXAMPLES

Catalysts Ru—SNS, Ru—PNN, SNS—RuHCl(CO), and PNN—RuHCl(CO) are commercially available from Johnson Matthey PLC and were used as supplied.

Ru-MACHO-BH is available from Strem Chemicals and was used as supplied.

OsPNN(N) is available from Merck and was used as supplied.

K3PO4 and NaOEt are available from Acros Organics, Alfa Aesar, Fischer Scientific, and Merck. K3PO4 was stored inside a glovebox filled with argon gas until use. Liquid reagents and anhydrous solvents were stored under nitrogen and transferred to reaction vials using standard air sensitive handling procedures.

Reactions were carried out in a Biotage Endeavour multi-well pressurised reaction system using 10 mL reaction vials.

### General Testing Procedure

The transition metal catalyst was added to the reaction vials along with the base in an argon filled glovebox. Anhydrous solvent was injected into the reaction vials, followed by the substrate via syringe. The reactions were sealed in the Biotage Endeavour multi-well pressurised reaction system. The reaction vials were purged with nitrogen five times, and purged with hydrogen once before being sealed and heated to the desired reaction temperature. The reaction vessels were pressurised with hydrogen to 400 psi with constant stirring at from 450 to 600 rpm. Each reaction was carried out for a period of 16 hours. The reaction mixtures were analysed by gas chromatography (GC) using ethanol as a solvent.

### Measurement Methods

Gas chromatography (GC) measurements were conducted using a Varian 3900 or 3800 gas chromatograph system. Unless otherwise indicated, reaction conversions were determined by GC analysis.

### Example 1: Ester Hydrogenation

The General Procedure described above was carried out on the following ester-containing substrates of Formula (I):

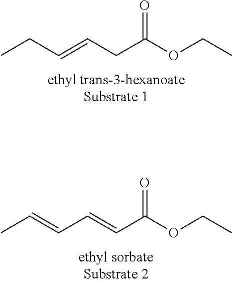

The desired products from Substrate 1 and Substrate 2 were the corresponding alcohols of Formula (II), namely:

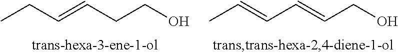

The base was varied between a “weak base” (e.g. K3PO4) according to the process of the presently disclosed subject matter, and a “strong base” not according to the process of the presently disclosed subject matter (e.g. NaOEt, or NaO′Bu)

Various reactions conditions were varied including the amount of base (relative to the substrate), substrate to transition metal catalyst, reaction temperature, solvent system, and transition metal catalyst. The transition metal catalysts used in the examples are as defined hereinabove. The results, as analysed by gas chromatography, show the conversion to the corresponding alcohol of Formula (II), the molar amount of unreacted starting material, the molar amount of saturated starting material (i.e. the instance where the double bond(s) of the starting material have been reduced but not the ester), and the molar amount of waxy ester by-products. The results of these experiments are summarised in Table 1 below.

Examples 1-10 and 17-48 are according to the presently disclosed subject matter, whilst Examples 11-16 and 49-56 are comparative examples.

TABLE 1

| | | | | | 1^{st} | | 2^{nd} | |
|---|---|---|---|---|---|---|---|---|
| Exp | | | | 1^{st} | Solvent | 2^{nd} | Solvent | |
| No. | Substrate | Catalyst | S/C | Solvent | (mL) | Solvent | (μL) | Base |
| 1 | 1 | OsPNN(N) | 1000 | MeTHF | 2 | — | — | K₃PO₄ |
| 2 | 1 | OsPNN(N) | 1000 | MeTHF | 2 | — | — | K₃PO₄ |
| 3 | 1 | OsPNN(N) | 1000 | — | — | — | — | K₃PO₄ |
| 4 | 1 | Ru-SNS | 5000 | PhMe | 3 | EtOH | 400 | K₃PO₄ |
| 5 | 1 | Ru-SNS | 5000 | PhMe | 3 | EtOH | 400 | K₃PO₄ |
| 6 | 1 | Ru-SNS | 1000 | PhMe | 2 | — | — | K₃PO₄ |
| 7 | 1 | Ru-SNS | 2000 | PhMe | 3 | — | — | K₃PO₄ |
| 8 | 1 | OsPNN(N) | 5000 | PhMe | 2 | — | — | K₃PO₄ |
| 9 | 1 | Ru-SNS | 2000 | PhMe | 2 | — | — | K₃PO₄ |
| 10 | 1 | Ru-SNS | 1000 | — | — | — | — | K₃PO₄ |
| 11 | 1 | Ru-SNS | 1000 | MeTHF | 1 | — | — | NaOEt |
| 12 | 1 | Ru-PNN | 1000 | MeTHF | 1 | — | — | NaOEt |
| 13 | 1 | OsPNN(N) | 1000 | MeTHF | 2 | — | — | NaOEt |
| 14 | 1 | SNS-Ru-HCl(CO) | 1000 | MeTHF | 2 | — | — | NaOEt |
| 15 | 1 | Ru-MACHO-BH | 1000 | MeTHF | 1 | — | — | NaOEt |
| 16 | 1 | Ru-PNN | 50000 | — | — | — | — | NaOEt |
| 17 | 2 | Ru-SNS | 1000 | PhMe | 3 | EtOH | 400 | K₃PO₄ |
| 18 | 2 | Ru-SNS | 1000 | PhMe | 3 | EtOH | 400 | K₃PO₄ |
| 19 | 2 | Ru-SNS | 2000 | PhMe | 3 | EtOH | 400 | K₃PO₄ |
| 20 | 2 | Ru-SNS | 5000 | PhMe | 5 | MeOH | 400 | K₃PO₄ |
| 21 | 2 | Ru-SNS | 5000 | PhMe | 5 | MeOH | 300 | K₃PO₄ |
| 22 | 2 | Ru-SNS | 5000 | PhMe | 5 | MeOH | 600 | K₃PO₄ |
| 23 | 2 | Ru-SNS | 5000 | PhMe | 5 | MeOH | 100 | K₃PO₄ |
| 24 | 2 | Ru-SNS | 5000 | PhMe | 3 | — | — | K₃PO₄ |
| 25 | 2 | Ru-SNS | 5000 | PhMe | 5 | MeOH | 200 | K₃PO₄ |
| 26 | 2 | Ru-SNS | 5000 | PhMe | 5 | EtOH | 400 | K₃PO₄ |
| 27 | 2 | Ru-SNS | 5000 | PhMe | 3 | ¹BuOH | 400 | K₃PO₄ |
| 28 | 2 | Ru-SNS | 5000 | PhMe | 5 | MeOH | 500 | K₃PO₄ |
| 29 | 2 | Ru-SNS | 5000 | PhMe | 3 | EtOH | 400 | K₃PO₄ |
| 30 | 2 | Ru-SNS | 5000 | PhMe | 5 | MeOH | 800 | K₃PO₄ |
| 31 | 2 | Ru-SNS | 5000 | PhMe | 5 | MeOH | 700 | K₃PO₄ |
| 32 | 2 | Ru-SNS | 2000 | PhMe | 3 | EtOH | 400 | K₃PO₄ |
| 33 | 2 | Ru-SNS | 1000 | PhMe | 3 | EtOH | 400 | K₃PO₄ |
| 34 | 2 | Ru-SNS | 5000 | PhMe | 5 | EtOH | 400 | K₃PO₄ |
| 35 | 2 | Ru-SNS | 5000 | MeTHF | 3 | EtOH | 400 | K₃PO₄ |
| 36 | 2 | Ru-SNS | 5000 | PhMe | 5 | EtOH | 700 | K₃PO₄ |
| 37 | 2 | Ru-SNS | 5000 | PhMe | 5 | ¹BuOH | 400 | K₃PO₄ |
| 38 | 2 | Ru-SNS | 5000 | PhMe | 3 | — | — | K₃PO₄ |
| 39 | 2 | Ru-SNS | 5000 | PhMe | 3 | ¹BuOH | 400 | K₃PO₄ |
| 40 | 2 | Ru-SNS | 1000 | PhMe | 3 | — | — | K₃PO₄ |
| 41 | 2 | Ru-SNS | 5000 | MeTHF | 3 | EtOH | 400 | K₃PO₄ |
| 42 | 2 | Ru-SNS | 5000 | PhMe | 3 | EtOH | 400 | K₃PO₄ |
| 43 | 2 | Ru-SNS | 1000 | PhMe | 3 | — | — | K₃PO₄ |
| 44 | 2 | Ru-SNS | 5000 | PhMe | 3 | EtOH | 400 | K₃PO₄ |
| 45 | 2 | Ru-SNS | 5000 | PhMe | 3 | EtOH | 400 | K₃PO₄ |
| 46 | 2 | Ru-SNS | 2000 | PhMe | 3 | — | — | K₃PO₄ |
| 47 | 2 | Ru-SNS | 5000 | PhMe | 5 | EtOH | 700 | K₃PO₄ |
| 48 | 2 | Ru-SNS | 5000 | PhMe | 3 | — | — | K₃PO₄ |
| 49 | 2 | Ru-SNS | 5000 | PhMe | 5 | EtOH | — | NaOEt |
| 50 | 2 | Ru-SNS | 5000 | PhMe | 5 | EtOH | 700 | NaO [symbol] Bu |
| 51 | 2 | Ru-SNS | 5000 | PhMe | 5 | EtOH | 700 | NaOEt |
| 52 | 2 | Ru-SNS | 5000 | PhMe | 5 | EtOH | 700 | NaOEt |
| 53 | 2 | Ru-SNS | 5000 | PhMe | 5 | EtOH | 700 | NaO [symbol] Bu |
| 54 | 2 | Ru-SNS | 5000 | PhMe | 5 | EtOH | 700 | NaO [symbol] Bu |
| 55 | 2 | Ru-SNS | 5000 | PhMe | 5 | EtOH | 700 | NaO [symbol] Bu |
| 56 | 2 | Ru-SNS | 5000 | PhMe | 5 | EtOH | 700 | NaOEt |

| | | | % | | | | |
|---|---|---|---|---|---|---|---|
| | | | | | Saturated | | Wax |
| | | | | Starting | Starting | By- | product |
| Exp | Base | Temp | | Material | Material | products | total |
| No. | (mol %) | (° C.) | Conversion | (%) | (%) | (%) | (%) |
| 1 | 10 | 40 | 100.0 | 0.0 | 0.0 | 0.0 | 0.0 |
| 2 | 10 | 40 | 96.1 | 0.0 | 0.0 | 0.0 | 3.9 |
| 3 | 10 | 40 | 95.0 | 0.0 | 0.0 | 0.0 | 5.0 |
| 4 | 40 | 70 | 92.1 | 0.0 | 0.0 | 1.6 | 6.2 |
| 5 | 40 | 70 | 91.7 | 0.0 | 0.0 | 1.2 | 7.1 |
| 6 | 40 | 70 | 82.4 | 0.0 | 1.2 | 10.1 | 6.3 |
| 7 | 40 | 70 | 81.2 | 0.0 | 0.0 | 2.7 | 16.1 |
| 8 | 40 | 40 | 79.5 | 4.3 | 7.1 | 0.0 | 9.3 |
| 9 | 40 | 70 | 79.0 | 0.0 | 0.0 | 3.6 | 17.5 |
| 10 | 40 | 70 | 76.8 | 1.3 | 2.9 | 14.1 | 4.9 |
| 11 | 10 | 40 | 28.8 | 0.0 | 0.0 | 47.6 | 23.6 |
| 12 | 10 | 40 | 0.0 | 0.0 | 0.0 | 91.3 | 8.7 |
| 13 | 10 | 40 | 18.6 | 54.5 | 26.9 | 0.0 | 0.0 |
| 14 | 10 | 40 | 17.1 | 60.7 | 14.0 | 8.3 | 0.0 |
| 15 | 10 | 40 | 9.7 | 88.5 | 1.8 | 0.0 | 0.0 |
| 16 | 50 | 40 | 0.0 | 0.0 | 1.8 | 2.9 | 95.3 |
| 17 | 40 | 50 | 64.1 | 4.1 | 0.8 | 29.5 | 1.6 |
| 18 | 40 | 60 | 61.2 | 0.0 | 0.7 | 14.0 | 24.0 |
| 19 | 40 | 50 | 59.9 | 9.4 | 0.9 | 17.8 | 12.0 |
| 20 | 40 | 70 | 55.1 | 9.4 | 12.5 | 6.2 | 16.9 |
| 21 | 40 | 70 | 54.4 | 3.8 | 10.5 | 15.5 | 15.7 |
| 22 | 40 | 70 | 54.4 | 11.9 | 12.7 | 5.2 | 15.7 |
| 23 | 40 | 70 | 54.0 | 3.4 | 11.2 | 10.2 | 21.3 |
| 24 | 40 | 70 | 53.7 | 1.9 | 3.1 | 39.0 | 2.3 |
| 25 | 40 | 70 | 52.5 | 5.9 | 13.3 | 8.5 | 19.8 |
| 26 | 40 | 70 | 52.4 | 10.1 | 15.0 | 1.1 | 21.4 |
| 27 | 40 | 70 | 52.2 | 4.3 | 3.7 | 37.3 | 2.6 |
| 28 | 40 | 70 | 52.0 | 12.0 | 12.9 | 15.2 | 7.9 |
| 29 | 40 | 70 | 51.3 | 5.8 | 3.3 | 23.3 | 16.3 |
| 30 | 40 | 70 | 51.2 | 17.0 | 11.1 | 8.8 | 12.0 |
| 31 | 40 | 70 | 50.3 | 18.3 | 11.1 | 8.8 | 11.6 |
| 32 | 40 | 70 | 50.2 | 0.0 | 5.3 | 14.2 | 30.3 |
| 33 | 40 | 70 | 48.9 | 0.0 | 1.2 | 18.5 | 31.4 |
| 34 | 40 | 70 | 48.8 | 11.0 | 3.4 | 23.5 | 13.4 |
| 35 | 40 | 70 | 48.5 | 1.0 | 9.8 | 12.9 | 27.8 |
| 36 | 40 | 70 | 48.0 | 14.9 | 13.2 | 9.6 | 14.2 |
| 37 | 40 | 70 | 47.7 | 5.2 | 11.8 | 13.5 | 21.8 |
| 38 | 40 | 60 | 47.6 | 8.3 | 2.0 | 39.5 | 2.7 |
| 39 | 40 | 70 | 47.3 | 2.6 | 10.4 | 13.5 | 26.2 |
| 40 | 40 | 60 | 47.1 | 0.0 | 0.0 | 47.2 | 5.7 |
| 41 | 40 | 70 | 46.9 | 3.0 | 4.7 | 40.8 | 4.3 |
| 42 | 40 | 60 | 46.7 | 18.6 | 2.2 | 27.6 | 4.9 |
| 43 | 40 | 70 | 46.4 | 0.0 | 0.7 | 11.2 | 41.8 |
| 44 | 40 | 70 | 45.9 | 8.9 | 3.4 | 37.3 | 4.6 |
| 45 | 40 | 70 | 45.5 | 10.8 | 14.4 | 10.3 | 19.0 |
| 46 | 40 | 50 | 45.0 | 18.6 | 15.4 | 11.7 | 9.3 |
| 47 | 40 | 80 | 44.8 | 12.3 | 18.0 | 4.0 | 20.9 |
| 48 | 40 | 70 | 44.8 | 4.6 | 3.3 | 43.7 | 3.7 |
| 49 | 20 | 80 | 31.5 | 6.7 | 19.9 | 12.6 | 29.3 |
| 50 | 20 | 80 | 31.5 | 7.5 | 22.1 | 23.0 | 25.9 |
| 51 | 50 | 80 | 28.3 | 5.7 | 20.2 | 9.3 | 36.5 |
| 52 | 10 | 80 | 25.7 | 20.4 | 19.2 | 18.8 | 15.9 |
| 53 | 50 | 80 | 14.5 | 8.6 | 25.5 | 5.2 | 46.2 |
| 54 | 10 | 80 | 4.9 | 3.2 | 4.2 | 4.3 | 83.4 |
| 55 | 5 | 80 | 2.5 | 92.8 | 1.2 | 2.8 | 0.8 |
| 56 | 5 | 80 | 1.5 | 93.7 | 0.6 | 2.8 | 1.3 |

[symbol]

indicates data missing or illegible when filed

The examples show that excellent conversion to the desired alcohol of Formula (II), with minimal by-product formation, can be achieved by the process of the presently disclosed subject matter. In particular, low catalyst loadings can be used in a variety of solvent systems.

In contrast to when a base according to the presently disclosed subject matter is used, when a ‘strong’ base, such as NaOEt or Nao′Bu is used, poor conversion the desired alcohol of Formula (II) is achieved. Instead, by-products are formed such as those where the alkene bond(s) have been reduced, those comprising waxy ester materials, or other unidentified by-products.

1. A process for the hydrogenation of an ester-containing substrate of Formula (I) to produce an alcohol of Formula (II), 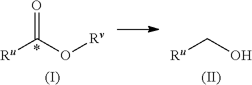
the process comprising treating the ester-containing substrate of Formula (I) with a base and a transition metal catalyst in the presence of molecular hydrogen, wherein:
the ester-containing substrate of Formula (I) comprises an a,b-g,d unsaturated ester or a b,g unsaturated ester;
R^{u} is an organic group having 3-70 carbon atoms, having the proviso that R^{u} is bonded to the carbonyl carbon (*) of the ester to form the a,b-g,d unsaturated ester or the b,g unsaturated ester of Formula (I);
R^{v} is an organic group having 1-70 carbon atoms; and
the conjugate acid of the base has a pKa of from 4 to 15.

2. A process according to claim 1, wherein R^{u} is an organic group having Formula (XII): 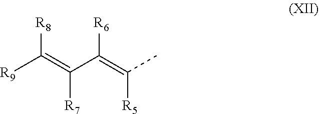
wherein the dashed bond indicates the bond to the carbonyl carbon (*) of the ester moiety of the ester-containing substrate of Formula (I); and
R₅-R₈ are each independently a hydrogen atom or an organic group having 1-70 carbon atoms, and
R₉ is H or an organic group having 1-70 carbon atoms.

3. A process according to claim 1, wherein R^{u} is an organic group having Formula (XIII): 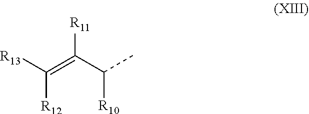
wherein the dashed bond indicates the bond to the carbonyl carbon (*) of the ester moiety of the ester-containing substrate of Formula (I); and
R₁₀-R₁₂ are each independently a hydrogen atom or an organic group having 1-70 carbon atoms, and
R₁₃ is H or an organic group having 1-70 carbon atoms.

4. A process according to claim 1, wherein R^{u} is an organic group having Formula (XII) or an organic group having formula (XIII) as defined in claim 2 or claim 3.

5. A process according to claim 1, wherein the ester-containing substrate of Formula (I) is an alkyl sorbate or an alkyl trans-3-hexanoate

6. A process according to claim 1 wherein the alcohol of Formula (II) is trans,trans-hexa-2,4-diene-1-ol, cis-hexa-3-ene-1-ol, or trans-hexa-3-ene-1-ol.

7. A process according to claim 1 wherein the conjugate acid of the base has a pKa of from 5 to 14, from 7 to 13, or from 9 to 12.

8. A process according to claim 1, wherein the base is a metal phosphate or a metal carbonate

9. A process according to claim 1, wherein the base is an alkali metal phosphate, an alkali earth metal phosphate, an alkali metal carbonate, or an alkali earth metal carbonate.

10. A process according to claim 1, wherein the base is an alkali metal phosphate, preferably lithium phosphate (Li₃PO₄), sodium phosphate (Na₃PO₄), potassium phosphate (K₃PO₄), or caesium phosphate (Cs₃PO₄).

11. A process according to claim 1, wherein the base is present in solid form.

12. A process according to claim 1, wherein the base is present in a range of 30 to 70 mol % based upon the total amount of ester-containing substrate, in a range of 30 to 60 mol % based upon the total amount of ester-containing substrate, or in a range of 30 to 50 mol % based upon the total amount of ester-containing substrate.

13. A process according to claim 1, wherein the transition metal catalyst comprises a tridentate ligand.

14. A process according to claim 13, wherein the transition metal catalyst comprises a tridentate ligand having a Formula (III) 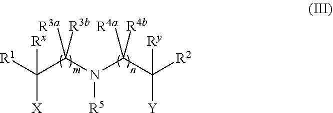
wherein:
X is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R¹ and one of R^{3a} and R^{3b} or R^{x} and one of R^{3a} and R^{3b} together with the atoms to which they are bound, form a ring;
or X is a heteroatom and when taken together with R¹ it forms an optionally substituted heterocycle when R^{x} is absent;
Y is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R² and one of R^{4a} and R^{4b} or R^{x} and one of R^{4a} and R^{4b} together with the atoms to which they are bound, form a ring;
or Y is a heteroatom and when taken together with R² it forms an optionally substituted heterocycle when R^{y} is absent;
R^{3a}, R^{3b}, R^{4a}, and R^{4b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R^{3a} and one of R^{4a} and R^{4b} or R^{3b} and one of R^{4a} and R^{4b} together with the atoms to which they are bound, form a heterocycle;
R⁵ is selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each m and n is independently 1 or 2; and
R^{a} and R^{b}, if present, are each independently selected from hydrogen, substituted or unsubstituted C₁₋₂₀-alkyl, substituted or unsubstituted C₂₋₂₀-alkenyl, substituted or unsubstituted C₂₋₂o-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when X and/or Y is —NR^{a}R^{b}, —PR^{a}R^{b}, —OPR^{a}R^{b}, or —NHPR^{a}R^{b}, R^{a} and R^{b} together with the heteroatom to which they are attached form a heterocycle

15. A process according to claim 1, wherein the transition metal catalyst has a Formula (IV) or Formula (V)
[M(L¹)(L²)_{d}] (IV)
[M(L¹)(L²)_{d}]W (V)
wherein:
M is a transition metal;
L¹ is a tridentate ligand as defined in claim 14;
L² are ligands which may be the same or different;
d is 1, 2 or 3; and
W is a non-coordinated anionic ligand.

16. A process according to claim 15, wherein M is a transition metal selected from Ru and Os, preferably Ru.

17. A process according to claim 15, wherein each L² is independently selected from —H, —CO, —CN, —P(R′)₃, —As(R′)₃, —CR′, —OR′, —O(C═O)R′, —NR′₂, halogen (e.g. —Cl, —Br, —I), and solvent wherein each R′ is independently selected from substituted or unsubstituted alkyl, substituted or unsubstituted alkenyl, substituted or unsubstituted alkynyl, substituted or unsubstituted heteroalkyl, substituted or unsubstituted alkoxy, substituted or unsubstituted cycloalkyl, substituted or unsubstituted heterocycloalkyl, substituted or unsubstituted aryl, and substituted or unsubstituted heteroaryl.

18. A process according to claim 1, wherein the transition metal catalyst is [chemical structure]
